SEQUENCE LISTING (1 sequence)

SEQ ID NO: 1 - DNA, 31 bp, Artificial Sequence
  Oligonucleotide capture probe
  modified_base (1)..(1): tri-ethylene glycol-DBCO
ttttttttttgaatgatacggcgaccaccga

(19) United States
(12) Patent Application Publication
(10) Pub. No.: US 2023/0374582 A1
(43) Pub. Date: Nov. 23, 2023

(54) METHOD COMPRISING LIGHT-EMITTING MARKER

(71) Applicant: Sumitomo Chemical Co., Ltd., Tokyo (JP)

(72) Inventors: Martin Humphries, Godmanchester (GB); Jonathan Behrendt, Godmanchester (GB); Ilaria Grizzi, Godmanchester (GB)

(73) Assignee: Sumitomo Chemical Co., Ltd., Tokyo (JP)

(21) Appl. No.: 18/031,330

(22) PCT Filed: Oct. 12, 2021
(86) PCT No.: PCT/EP2021/078145

§ 371 Date: Apr. 11, 2023

(30) Foreign Application Priority Data
Oct. 12, 2020 (GB) 2016168.3
Mar. 11, 2021 (GB) 2103405.3

(51) Int. Cl. C12Q 1/6869; G01N 33/58

(52) U.S. Cl. CPC C12Q 1/6869; G01N 33/582

(57) ABSTRACT

A method of determining whether a test nucleotide comprises a base complementary to the next base of a template strand immediately downstream of a primer in a primed template nucleic acid molecule is described. The method comprises the use of a marked nucleotide comprising a test nucleotide conjugated to a light emitting marker by a linker.

### BACKGROUND

The present disclosure relates to methods for sequencing nucleic acids and reagents for use in such methods.

One of the technologies that have improved the study of nucleic acids is the development of fabricated arrays of immobilised nucleic acids. These arrays consist typically of a high-density matrix of polynucleotides immobilised onto a solid support material. Using such arrays, current sequencing methods allow for the parallel processing of millions or even billions of cloned nucleic acids or nucleic acid fragments in a single sequencing run. These high-throughput approaches to nucleic acid analysis are often referred to as massive parallel sequencing, or next generation sequencing (NGS) methods. NGS technologies differ in precise methodology and sequencing chemistry but share the feature of the parallel analysis of clonally amplified nucleic acid template clusters that are spatially separated and immobilised, for example, within a flow cell.

One way of determining the nucleotide sequence of a nucleic acid bound to an array is called “sequencing by synthesis” or “SBS”. This technique requires the incorporation of the correct nucleotide complementary to that of the nucleic acid being sequenced. Thus, each nucleotide residue is identified as it is incorporated into the growing nucleic acid strand. The incorporated nucleotide is read using an appropriate label attached thereto before removal of the label moiety and the subsequent next round of sequencing. Detection of the label can be carried out using various methods, including fluorescence spectroscopy or by other optical means. Generally, the preferred label is a fluorophore, which, after absorption of energy, emits radiation at a defined wavelength. Many fluorescent labels are known, for use in various different sequencing methods (see, for example, Anderson et al., Nano Lett. 2010, 10, 788-792, and U.S. Pat. No. 9,045,798).

Nevertheless, nucleotide detection remains a weak link in sequencing processes and improvements in nucleotide detection methods are required.

### SUMMARY

A summary of aspects of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects and/or a combination of aspects that may not be set forth.

The present inventors have found that using certain light-emitting markers to mark nucleotides in methods for analysing and/or sequencing nucleic acids can provide a high signal to noise ratio, allowing sequencing to be performed on smaller clone sizes and longer sequences.

Accordingly, in some embodiments there is provided a method of determining whether a test nucleotide comprises a base complementary to the next base of a template strand immediately downstream of a primer in a primed template nucleic acid molecule. The method comprises the steps of:
(a) providing a primed template nucleic acid molecule;
(b) providing a marked nucleotide comprising the test nucleotide conjugated to a light emitting marker by a linker, wherein the light emitting marker comprises a light emitting polymer or a light-emitting particle;
(c) contacting the primed template nucleic acid molecule with a reaction mixture that comprises a polymerase and one or more different test nucleotide(s), to thereby incorporate a test nucleotide into the primed strand only if the test nucleotide comprises a base complementary to the next base of the template strand; and
(d) detecting light emitted by the light emitting marker, wherein the detection of light identifies the incorporation of the test nucleotide into the primed strand, and thereby indicates that the test nucleotide comprises a base complementary to the next base of the template strand;
   wherein step (b) occurs before step (c), such that the test nucleotide is present in the reaction mixture in the form of a marked nucleotide, or
   wherein step (c) occurs before step (b), such that the marked nucleotide comprising the test nucleotide is formed after incorporation of the test nucleotide into the primed strand.

Optionally, step (c) occurs before step (b), and the method comprises:
(a) providing a primed template nucleic acid molecule;
(b) contacting the primed template nucleic acid molecule with a reaction mixture that comprises a polymerase and one or more different test nucleotides, to thereby incorporate a test nucleotide into the primed strand of the primed template nucleic acid molecule only if the test nucleotide comprises a base complementary to the next base of the template strand;
(c) conjugating the test nucleotide to a light emitting marker using a linker to form a marked nucleotide, wherein the light emitting marker comprises a light emitting polymer or a light-emitting particle; and
(d) detecting light emitted by the light emitting marker, wherein the detection of light identifies the incorporation of the test nucleotide into the primed strand, and thereby indicates that the test nucleotide comprises a base complementary to the next base of the template strand.

Optionally, the method is performed using an array comprising clusters of cloned template fragments, each cloned template fragment representing a primed template nucleic acid molecule. Optionally, each cluster comprises less than 1000 primed template nucleic acid molecules, such as less than 500, 250, 100, 50, or less than 10 primed template nucleic acid molecules.

Optionally, the method does not comprise the use of clusters of cloned template fragments, and the method comprises the use of a single copy of each primed template nucleic acid molecule.

Optionally, the linker is a cleavable linker and the method further comprises step (e) cleaving the linker to dissociate the light emitting marker from the test nucleotide.

Optionally, the linker comprises a flexible spacer.

Optionally, the linker comprises or further comprises a stable complex between a ligand and a biomolecule. The biomolecule may be a protein.

Optionally, the ligand is an antigen and the biomolecule comprises an antigen-binding fragment. The biomolecule may be an antibody.

Optionally, the ligand is biotin and the biomolecule is selected from avidin, streptavidin, neutravidin and recombinant variants thereof.

Optionally, the light-emitting marker is a light-emitting particle having a light-emitting core containing or consisting of the light-emitting polymer.

Optionally, the light-emitting core contains the light-emitting polymer and a matrix material. The matrix material may be silica.

Optionally, the light-emitting particle has a coating. The coating may comprise polyethylene glycol.

Optionally, the light emitting marker comprises a soluble light emitting polymer.

Optionally, the nucleotide further comprises a terminator or reversible terminator moiety.

Optionally, the light emitting marker has a brightness of at least 3×10⁶ cm⁻¹M⁻¹.

Preferably, the light emitting marker has a brightness of at least 5×10⁶, 8×10⁶, 1×10⁷, 2×10⁷, 3×10⁷, or 4×10⁷ cm⁻¹M⁻¹.

Optionally, the nucleotide is a cleavable oligonucleotide for use in sequencing-by-ligation methods.

Optionally, the reaction mixture comprises a single species of test nucleotide.

Optionally, the method is a method of sequencing-by-synthesis.

Optionally, the test nucleotide is attached to a ligand by a cleavable linker, and the light emitting marker comprises a biomolecule that is capable of forming a stable complex with the ligand, wherein the marked nucleotide is formed by contacting the ligand with the biomolecule thereby forming a stable complex between the ligand and biomolecule.

Optionally, contacting the ligand with the biomolecule to form a stable complex occurs after incorporation of the test nucleotide into the primed strand.

Optionally, the test nucleotide is attached to the light emitting marker by a cleavable linker.

Optionally, the test nucleotide is present in the reaction mixture in the form of a marked nucleotide comprising the test nucleotide attached to the light emitting marker by a cleavable linker.

Optionally, the reaction mixture comprises a plurality of different species of test nucleotides, such as two, three, or four different species of test nucleotides, each comprising a different light emitting marker arranged to emit light at a different wavelength. Detecting light emitted by the light emitting marker of one of the plurality of different species of test nucleotides identifies the incorporation of that particular test nucleotide into the primed strand, and thereby indicates that the particular test nucleotide comprises a base complementary to the next base of the template strand.

In some embodiments there is provided a marked nucleotide comprising the test nucleotide attached to the light emitting marker by a cleavable linker.

Optionally, the linker is a cleavable linker and the cleavable linker is attached to either or both of the test nucleotide and the light-emitting marker.

Optionally, when the light-emitting marker is a particulate light-emitting marker, the cleavable linker is attached to the light-emitting material of the particulate light-emitting marker

Optionally, when the light-emitting marker is a particulate light-emitting marker, the cleavable linker is attached to a surface or matrix of the particulate light-emitting marker.

Optionally, the cleavable linker is attached to the light-emitting marker by a stable complex formed between a ligand and a biomolecule.

Optionally, the cleavable linker is attached to the test nucleotide by a stable complex formed between a ligand and a biomolecule.

Optionally, the cleavable linker comprises a cleavable moiety as described herein.

Optionally, the light-emitting marker is a light-emitting particle having a light-emitting core containing or consisting of the light-emitting polymer.

Optionally, the light-emitting core contains the light-emitting polymer and a matrix material. The matrix material may be silica.

Optionally, the light-emitting particle has a coating. The coating may comprise polyethylene glycol.

Optionally, the light emitting marker comprises a soluble light emitting polymer.

Optionally, the nucleotide further comprises a terminator or reversible terminator moiety.

Optionally, the nucleotide is cleavable oligonucleotide for use in sequencing-by-ligation methods.

### DESCRIPTION OF THE DRAWINGS

The present disclosure is described in conjunction with the appended figures. It is emphasized that, in accordance with the standard practice in the industry, various features are not drawn to scale. In fact, the dimensions of the various features may be arbitrarily increased or reduced for clarity of discussion.

In the appended figures, similar components and/or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

The invention will now be described in more detail with reference to the drawings wherein:

FIG. 1 is a schematic illustration of a method of sequencing-by-synthesis according to some embodiments:

FIG. 2 is a schematic illustration of a method of sequencing-by-synthesis according to some embodiments.

FIG. 3 is two graphs showing the fluorescence intensities of detection reactions probing for decreasing quantities of surface target (Biotinylated BSA) using light-emitting markers. The total combined concentration of Bt-BSA and BSA is kept at 50 μg/ml, with the ratio of Bt-BSA to BSA indicated by % (e.g. 33% Bt-BSA/BSA represents 16.6 μg/ml Bt-BSA and 33.4 μg/ml BSA).

FIG. 4 is two graphs showing signal to noise ratios of detection reactions probing for decreasing quantities of surface target (Biotinylated BSA) using light-emitting markers. The total combined concentration of Bt-BSA and BSA is kept at 50 μg/ml, with the ratio of Bt-BSA to BSA indicated by % (e.g. 33% Bt-BSA/BSA represents 16.6 μg/ml Bt-BSA and 33.4 μg/ml BSA).

FIG. 5 is two graphs showing the fluorescence intensities of detection reactions probing for decreasing quantities of surface target (Biotinylated BSA) using FITC. The total combined concentration of Bt-BSA and BSA is kept at 50 μg/ml, with the ratio of Bt-BSA to BSA indicated by % (e.g. 33% Bt-BSA/BSA represents 16.6 μg/ml Bt-BSA and 33.4 μg/ml BSA).

FIG. 6 is two graphs graph showing signal to noise ratios of detection reactions probing for decreasing quantities of surface target (Biotinylated BSA) using FITC. The total combined concentration of Bt-BSA and BSA is kept at 50 μg/ml, with the ratio of Bt-BSA to BSA indicated by % (e.g. 33% Bt-BSA/BSA represents 16.6 μg/ml Bt-BSA and 33.4 μg/ml BSA).

FIG. 7 is two graphs comparing the fluorescence intensities of detection reactions using light-emitting markers versus FITC, for decreasing quantities of surface target (Biotinylated BSA). The total combined concentration of Bt-BSA and BSA is kept at 50 μg/ml, with the ratio of Bt-BSA to BSA indicated by % (e.g. 33% Bt-BSA/BSA represents 16.6 μg/ml Bt-BSA and 33.4 μg/ml BSA).

FIG. 8 is two graphs showing signal to noise ratios of detection reactions using light-emitting markers versus FITC, for decreasing quantities of surface target (Biotinylated BSA). The total combined concentration of Bt-BSA and BSA is kept at 50 μg/ml, with the ratio of Bt-BSA to BSA indicated by % (e.g. 33% Bt-BSA/BSA represents 16.6 μg/ml Bt-BSA and 33.4 μg/ml BSA).

FIG. 9 is a graph showing the relative signal to noise ratios of detection reactions using light-emitting nanoparticles versus FITC. The total combined concentration of Bt-BSA and BSA is kept at 50 μg/ml, with the ratio of Bt-BSA to BSA indicated by % (e.g. 33% Bt-BSA/BSA represents 16.6 μg/ml Bt-BSA and 33.4 μg/ml BSA).

FIG. 10 is a diagram showing the experimental method used in Example 2.

FIG. 11 is a graph showing corrected fluorescent signal intensity versus DNA concentration using light-emitting nanoparticles versus FITC.

FIG. 12 is an enlarged version of a part of the graph shown in FIG. 11.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements. Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list.

The teachings of the technology provided herein can be applied to other systems, not necessarily exclusively the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The disclosed methods may comprise sequencing template fragments derived from a target nucleic acid.

Unless defined otherwise, all technical and scientific terms used herein have the same meaning as is commonly understood by one of ordinary skill in the art. For clarity, the following specific terms have the specified meanings.

The term “nucleic acid” can refer to at least two nucleotide monomers linked together. Examples include, but are not limited to DNA, such as genomic or cDNA; RNA, such as mRNA, sRNA or rRNA; or a hybrid of DNA and RNA. Thus, a “nucleic acid” is a polynucleotide, such as DNA, RNA, or any combination thereof, that can be acted upon by a polymerizing enzyme during nucleic acid synthesis. The term “nucleic acid” includes single-, double-, or multiple-stranded DNA, RNA and analogs (derivatives) thereof. As apparent from the disclosure below and elsewhere herein, a nucleic acid can have a naturally occurring nucleic acid structure or a non-naturally occurring nucleic acid analog structure. A nucleic acid can contain phosphodiester bonds; however, in some embodiments, nucleic acids may have other types of backbones, comprising, for example, phosphoramide, phosphorothioate, phosphorodithioate, O-methylphosphoroamidite and peptide nucleic acid backbones and linkages. Nucleic acids can have positive backbones; non-ionic backbones, and non-ribose based backbones. Nucleic acids may also contain one or more carbocyclic sugars. The nucleic acids used in methods or compositions herein may be single stranded or, alternatively double stranded, as specified. In some embodiments a nucleic acid can contain portions of both double stranded and single stranded sequence, for example, as demonstrated by forked adapters. A nucleic acid can contain any combination of deoxyribo- and ribo-nucleotides, and any combination of bases, including uracil, adenine, thymine, cytosine, guanine, inosine, xanthanine, hypoxanthanine, isocytosine, isoguanine, and base analogs such as nitropyrrole (including 3-nitropyrrole) and nitroindole (including 5-nitroindole), etc.

A “template nucleic acid” is a nucleic acid to be detected or sequenced using any sequencing method disclosed herein. As used herein, a “primed template nucleic acid” (or alternatively, “primed template nucleic acid molecule”) is a template nucleic acid primed with (i.e., hybridized to) a primer, wherein the primer is an oligonucleotide having a 3′-end with a sequence complementary to a portion of the template nucleic acid. The primer can optionally have a free 5′-end (e.g., a portion of the primer being non-hybridized with the template), be fully hybridized to the template or can be continuous with the template (e.g., via a hairpin structure). The primed template nucleic acid includes the complementary primer and the template nucleic acid to which it is bound. Unless explicitly stated, a primed template nucleic acid can have either a 3′-end that is extendible by a polymerase, or a 3′-end that is blocked from extension. In preferred embodiments, genomic DNA fragments, or amplified copies thereof, are used as the target nucleic acid. In other preferred embodiments, mitochondrial or chloroplast DNA is used. Other embodiments are targeted to RNA or derivatives thereof such as mRNA or cDNA.

The term “nucleotide sequence” is intended to refer to the order and type of nucleotide monomers in a nucleic acid polymer. A nucleotide sequence is a characteristic of a nucleic acid molecule and can be represented in any of a variety of formats including, for example, a depiction, image, electronic medium, series of symbols, series of numbers, series of letters, series of colors, etc. A series of “A,” “T,” “G,” and “C” letters is a well-known sequence representation for DNA that can be correlated, at single nucleotide resolution, with the actual sequence of a DNA molecule. A similar representation is used for RNA except that “T” is replaced with “U” in the series.

A “nucleotide” is a molecule that includes a nitrogenous base, a five-carbon sugar (ribose or deoxyribose), and at least one phosphate group. The term embraces, but is not limited to, ribonucleotides, deoxyribonucleotides, nucleotides modified to include exogenous labels or reversible terminators, and nucleotide analogs. The test nucleotide is preferably a native nucleotide. A “native” nucleotide refers to a naturally occurring nucleotide that does not include an exogenous label (e.g., a fluorescent dye, or other label) or chemical modification such as may characterize a nucleotide analog. The term “dNTP” refers to any deoxyribonucleotide triphosphate, and a dNTP for use in the disclosed method may comprise a native nucleotide. Examples of native nucleotides that may be used as a test nucleotide in the disclosed methods include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate). The test nucleotide may be nucleotide analog. A “nucleotide analog” has one or more modifications, such as chemical moieties, which replace, remove and/or modify any of the components (e.g., nitrogenous base, five-carbon sugar, or phosphate group(s)) of a native nucleotide. Nucleotide analogs may be either incorporable or non-incorporable by a polymerase in a nucleic acid polymerization reaction. Optionally, the 3′—OH group of a nucleotide analog is modified with a moiety. The moiety may be a 3′ reversible or irreversible terminator of polymerase extension. The base of a nucleotide may be any of adenine, cytosine, guanine, thymine, or uracil, or analogs thereof. Optionally, a nucleotide has an inosine, xanthine, hypoxanthine, isocytosine, isoguanine, nitropyrrole (including 3-nitropyrrole) or nitroindole (including 5-nitroindole) base. Nucleotides may include, but are not limited to, ATP, UTP, CTP, GTP, ADP, UDP, CDP, GDP, AMP, UMP, CMP, GMP, dATP, dTTP, dUTP, dCTP, dGTP, dADP, dTDP, dCDP, dGDP, dAMP, dTMP, dCMP, and dGMP. Nucleotides may also contain terminating inhibitors of DNA polymerase, dideoxynucleotides or 2′,3′ dideoxynucleotides, which are abbreviated as ddNTPs (ddGTP, ddATP, ddTTP, ddUTP and ddCTP).

The “next correct nucleotide” (also referred to as the “cognate” nucleotide) refers to the nucleotide type that will bind and/or incorporate at the 3′ end of a primer to complement a base in a template strand to which the primer is hybridized. The base in the template strand is referred to as the “next template nucleotide” and is immediately 5′ of the base in the template that is hybridized to the 3′ end of the primer. The next correct nucleotide can be, but need not necessarily be, capable of being incorporated at the 3′ end of the primer or 3′ end of the nascent growing strand. A nucleotide having a base that is not complementary to the next template base is referred to as an “incorrect” (or “non-cognate”) nucleotide. A “marked nucleotide” refers to a nucleotide conjugated to any marker (e.g. a fluorophore) by a linker, wherein the nucleotide may or may not be incorporated into the primed strand.

A “polymerase” refers to any nucleic acid synthesizing enzyme, including but not limited to, DNA polymerase, RNA polymerase, reverse transcriptase, primase and transferase. Typically, the polymerase includes one or more active sites at which nucleotide binding and/or catalysis of nucleotide polymerization may occur. The polymerase may catalyze the polymerization of nucleotides to the 3′-end of a primer bound to its complementary nucleic acid strand. For example, a polymerase can catalyze the addition of a next correct nucleotide to the 3′ oxygen of the primer via a phosphodiester bond, thereby chemically incorporating the nucleotide into the primer. Optionally, the polymerase used in the provided methods is a processive polymerase. Optionally, the polymerase used in the provided methods is a distributive polymerase. Optionally, a polymerase need not be capable of nucleotide incorporation under one or more conditions used in a method set forth herein. For example, a mutant polymerase may be capable of forming a ternary complex but incapable of catalyzing nucleotide incorporation.

The term “providing”, as used, for example, in relation to a test nucleotide, a marked nucleotide, a template, a primer, or a primed template nucleic acid, refers to the preparation and delivery of one or many of the relevant reagents, for example to a reaction mixture or reaction chamber.

The term “contacting” refers to the mixing together of reagents (e.g., mixing a primed template nucleic acid molecule with a reaction mixture that comprises a polymerase and the test nucleotide) so that a physical binding reaction or a chemical reaction may take place.

The term, “incorporating” or “chemically incorporating” refers to the inclusion of the cognate nucleotide, for example, by correct base pairing with the corresponding base in the template strand, and by attachment to the primer by formation of a phosphodiester bond. Accordingly, the term “incorporating” refers to the process of joining a nucleotide to the 3′-end of a primer by formation of a phosphodiester bond. Thus, the incorporation of a nucleotide at the 3′ end of the primer leads to extension of the primer. The incorporated nucleotide thereby provides the 3′ end of the primer in the subsequent sequencing cycle. The 3′ end of the primer thereby advances by one position along the template strand in each sequencing cycle.

As used herein, “extension” refers to the process in which a polymerase enzyme catalyzes addition of one or more nucleotides at the 3′-end of the primer, thereby leading to extension of the primer.

In some embodiments, the sequencing method may comprise sequencing-by-synthesis (SBS) method. In some embodiments, a SBS method may comprise four steps:
1. library preparation;
2. cluster generation;
3. sequencing; and
4. data analysis.

Advantageously, in some embodiments, as will be apparent from the below discussion, the method disclosed herein may not require the step of cluster generation, and thus the disclosed methods may comprise three steps: library preparation, sequencing, and data analysis.

1. Library Preparation

Library preparation is a molecular biology protocol that converts a nucleic acid template, such as a genomic DNA sample, or cDNA sample, into a sequencing library, which can then be sequenced, for example, using a Next Generation Sequencing (NGS) instrument.

A target nucleic acid sample can, in some embodiments, be processed prior to performing other modifications. For example, a target nucleic acid sample can be amplified prior to attaching to a bead, or prior to attaching to the surface of a solid support.

Amplification is particularly useful when samples are in low abundance or when small amounts of a target nucleic acid are provided. Methods that amplify the vast majority of sequences in a genome are referred to as “whole genome amplification” methods. Examples of such methods include multiple displacement amplification (MDA), strand displacement amplification (SDA), or hyperbranched strand displacement amplification, each of which can be carried out using degenerate primers. Particularly useful methods are those that are used during sample preparation methods recommended by commercial providers of whole genome sequencing platforms (e.g. Illumina Inc., San Diego and Life Technologies Inc., Carlsbad).

The sequencing library may be prepared by random fragmentation of the nucleic acid sample. The term “fragment,” when used in reference to a first nucleic acid, is intended to mean a second nucleic acid consisting of a part or portion of the sequence of the first nucleic acid.

In some embodiments, fragmentation inherently results from amplification, for example, in cases where the portion of the template that occurs between sites where flanking primers hybridize is selectively copied.

In other embodiments, fragmentation may be achieved using chemical, enzymatic or physical techniques known in the art.

As discussed below, an advantage of the disclosed method is that smaller clusters of nucleotides are required, allowing tracking of individual errors as they occur. As such, with compensation this allows longer template fragments to be read than is possible using current sequencing methods. Fragments in a desired size range can be obtained using separation methods known in the art such as gel electrophoresis. Fragmentation can be carried out to obtain template nucleic acid fragments that have a minimum size of at least about 0.1 kb, 0.5 kb, 1 kb, 2 kb, 3, kb, 4 kb, 5 kb, 10 kb or longer in length.

Adapters, which may be referred to as “library adapters” may be ligated to the template fragments, such as, for example, ligation of 5′ and 3′ adapters to each DNA fragment. “Tagmentation” may be used to combine the fragmentation and ligation reactions into a single step that may increase the efficiency of the library preparation process.

Adapter-ligated fragments may be amplified and purified by any suitable method currently used in the art. For example, adapter-ligated fragments may be PCR amplified and gel purified.

The fragments that are produced from one or more nucleic acid templates can be captured randomly at locations on a solid support surface.

Solid supports can be two-or three-dimensional and can be a planar surface (e.g., a glass slide) or can be shaped. Useful materials include glass (e.g., controlled pore glass (CPG)), quartz, plastic (such as polystyrene (low cross-linked and high cross-linked polystyrene), polycarbonate, polypropylene and poly(methylmethacrylate)), acrylic copolymer, polyamide, silicon, metal (e.g., alkanethiolate-derivatized gold), cellulose, nylon, latex, dextran, gel matrix (e.g., silica gel), polyacrolein, or composites. Suitable three-dimensional solid supports include, for example, spheres, microparticles, beads, membranes, slides, plates, micromachined chips, tubes (e.g., capillary tubes), microwells, microfluidic devices, channels, filters, or any other structure suitable for anchoring a nucleic acid. Solid supports can include planar microarrays or matrices capable of having regions that include populations of nucleic acids or primers. Examples include nucleoside-derivatized CPG and polystyrene slides; derivatized magnetic slides; polystyrene grafted with polyethylene glycol, and the like.

A solid support to which nucleic acids may be attached in the sequencing method have a continuous or monolithic surface. Thus, fragments can attach at spatially random locations wherein the distance between nearest neighbor fragments (or nearest neighbor clusters derived from the fragments) may be variable. The resulting arrays may have a variable or random spatial pattern of features.

Different template fragments that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features or locations on the same substrate. Exemplary sites include, for example, wells in a substrate, beads (or other particles) in or on a substrate, projections from a substrate, ridges on a substrate or channels in a substrate. The sites of an array can be separate substrates each bearing a different molecule. Exemplary arrays in which separate substrates are located on a surface include, for example, those having beads in wells.

As discussed below, an advantage provided by the disclosed method is that smaller clusters of template fragments can be used than is possible using current sequencing methods. In some embodiments it is possible to sequence single copy template fragments. As a result, the disclosed methods can advantageously use arrays having a high density of features such as, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², 10⁷ features/cm², 5×10⁷ features/cm², 10⁸ features/cm², 5×10⁸ features/cm², 10⁹ features/cm², 5×10⁹ features/cm², or higher.

Flow cells provide a convenient format for housing an array of nucleic acid fragments for use in the disclosed methods. As used herein, the term “flow cell” is intended to mean a chamber having a surface across which one or more fluid reagents can be flowed. Generally, a flow cell will have an ingress opening and an egress opening to facilitate flow of fluid. Flow cells provide a convenient format for use in the disclosed method that involves repeated delivery of reagents in cycles. For example, to initiate a first SBS cycle, one or more dNTPs, DNA polymerase, etc., can be flowed into/through a flow cell that houses an array of nucleic acid fragments. Washes can easily be carried out in the flow cell between the various delivery steps. The cycle can be repeated n times to extend the primer by n nucleotides, thereby detecting a sequence of length n.

For cluster generation, the library of adapter-ligated template fragments may be loaded into a flow cell where fragments are captured on a lawn of surface-bound binding molecules, such as oligonucleotides complementary to the library adapters.

2. Cluster Generation

Cluster generation is a process of clonal amplification of target nucleic acid templates which may be used or required, for example, for imaging systems which cannot detect single fluorescence events. Various suitable methods for clonally amplifying nucleic acid template molecules to produce clusters of cloned template will be known to the skilled person. Any suitable method may be used and typically, these methods comprise polymerase chain reaction (PCR)-based techniques. The cluster generation procedure is relatively complicated and time-consuming, and may introduce errors into the cloned template nucleic acids.

In the cluster generation step, each template fragment is clonally amplified into distinct clusters. The result is a clonal grouping of identical template fragments bound to the surface of the flow cell. Each cluster on the flow cell produces a single sequencing read.

For example, 10,000 clusters on the flow cell would produce 10,000 sequence reads. Where paired-end reads are implemented, a second read of each sequence would also be performed.

Each cluster is seeded by a single template nucleic acid fragment and is clonally amplified, for example, using a PCR-based approach, such as involving the use of forward and reverse primers that are attached to the support within the flow cell. In current sequencing methods, a typical cluster has in the order of 1000 copies.

To enable the generation of defined clusters, the template fragments may be captured onto surfaces that are patterned for example, with embedded beads (typically 1-2 μm in diameter) or wells (typically 200-600 nm in diameter). Each bead or well only captures a single template fragment and the size of the bead or well defines the maximum size of the cluster. The structured organization provided by the patterned surface of the flow cell provides improved, regular spacing of template clusters, and increased cluster density, which provides advantages over non-pattered clusters, such as in relation to signal detection.

Bridge amplification may be used to generate clusters. Bridge amplification may occur on the surface of the flow cell. For example, in currently used methods, the surface of the flow cell is coated with a “lawn” of oligonucleotides. In the first step of bridge amplification, a single-stranded sequencing library (with complementary adapter ends) is loaded into the flow cell. Individual molecules in the library bind to complementary oligos as they “flow” across the oligo lawn. Priming occurs as the opposite end of a ligated fragment bends over and “bridges” to another complementary oligo on the surface. Repeated denaturation and extension cycles (similar to PCR) result in localized amplification of single molecules into millions of unique, clonal clusters across the flow cell.

Other suitable amplification methods known in the art can also be used to produce immobilized amplicons from immobilized nucleic acid fragments. For example one or more clusters can be formed via solid-phase PCR, solid-phase MDA, solid-phase RCA etc., whether one or both primers of each pair of amplification primers are immobilized.

Errors occurring during cluster generation, as a result of the amplification procedures such as PCR, contribute to sequencing errors and can cause problems for genetic analysis. For example, several different types of errors may occur during PCR, from single-nucleotide base substitutions to large-scale template-switching events. As PCR errors are propagated during exponential amplification, seemingly infrequent events can have profound implications for downstream analysis, particularly for next-generation sequencing. In the disclosed methods, such errors may be reduced by the use of smaller clusters containing fewer copies of the cloned template molecules and requiring fewer amplification cycles, or eradicated completely by the use of single-copy templates, which does not require any template amplification procedure.

Single molecule templates may be immobilized on solid supports using various methods that will be known to the skilled person, for example, by priming and extending single-stranded, single-molecule templates from immobilized primers. The use of single molecule templates in the disclosed methods potentially allows the analysis of much larger nucleic acids templates than is possible with clonally amplified clusters, and potentially longer read lengths.

The number of cloned template fragments available for sequencing (i.e. the size of the cluster), is proportional to the total level of fluorescence emitted by the labelled nucleotides that are incorporated into the cluster of template fragments during each sequencing cycle.

In current sequencing methods, each labelled dNTP is associated with a single fluorophore. Thus, in current sequencing methods, cluster generation is required because the fluorescence from one labelled dNTP is insufficient to detect above background. The formation of a cluster of template fragment clones is typically required to provide a significant and sustained detectable fluorescent signal.

In practice, the size of the cluster may be limited by the size of the bead or well that is used as the substrate in the sequencing reaction, in which each bead or well may comprise a single cluster of template fragment clones. The use of smaller clusters provided by the disclosed methods, therefore, potentially allows the use of substrates having a smaller surface area, and thus provides the possibility of an increased number of substrates (i.e. sequencing reads) per unit area of the flow cell or other solid support.

The present disclosure provides sequencing methods involving the use of marked nucleotides, comprising a test nucleotide conjugated to a light emitting marker by a linker. The disclosed light emitting markers have higher brightness (Where brightness=[extinction coefficient]×[photoluminescence quantum yield]) than the fluorophores that are currently used to label nucleotides.

The fluorescence output of a given fluorophore depends on the efficiency with which it absorbs and emits photons, and its ability to undergo repeated excitation/emission cycles. Absorption and emission efficiencies are most usefully quantified in terms of the molar extinction coefficient (EC) for absorption and the quantum yield (QY) for fluorescence. Both are constants under specific environmental conditions.

The value of EC is specified at a single wavelength (usually the absorption maximum), whereas QY is a measure of the total photon emission over the entire fluorescence spectral profile.

Fluorescence intensity per fluorophore (i.e. “brightness”) is proportional to the product of EC (at the relevant excitation wavelength) and QY.

Due to the high fluorescence intensity and low signal to noise ratio of the light emitting markers used in the disclosed method, clusters of cloned template fragment nucleic acids can be far smaller than those used in current methods.

For example, in some embodiments, sequencing methods comprising the disclosed light emitting markers may comprise the use of clusters of cloned template fragment nucleic acids comprising less than or about 100 template fragments, less than or about 50 template fragments, less than or about 30 template fragments, less than or about 20 template fragments, less than or about 10 template fragments, or less than or about 5 template fragments.

In some embodiments, the disclosed sequencing methods may comprise the use of a single template fragment nucleic acid. In such embodiments, the disclosed sequencing methods do not require a cluster generation step.

The use of smaller clusters than has previously been possible, or the use of a single template fragment, provides a number of advantages to the disclosed sequencing methods. For example, the amplification procedure (and thus the sequencing method as a whole) is simplified, with a reduction in time, cost, and complexity. There is also a reduced possibility of errors occurring as a result of the clonal amplification process.

Another advantage is that the clusters can be smaller and therefore more clusters can be incorporated onto an array or substrate.

3. Sequencing

In some embodiments, the disclosed method comprises the detection of single nucleotides as they are incorporated into template strands.

In some embodiments, nucleotides are added to a nucleic acid primer thereby extending the primer in a template-dependent manner. Detection of the order and type of nucleotides added to the primer can be used to determine the sequence of the template.

In current sequencing methods, each nucleotide comprises an associated fluorescent label that can be used to detect and identify the nucleotide. In the disclosed methods, a marked nucleotide, which comprises the test nucleotide conjugated by a linker to a light emitting marker is used. The fluorescence intensity of the light emitting markers of the disclosed method is much higher than that of the fluorophores used in current sequencing methods, and the signal to noise ratio is much lower.

During each sequencing cycle, a single labeled nucleotide, which is the next complementary nucleotide (i.e. comprises a base complementary to the next base of the template strand), is incorporated into the nucleic acid chain in a template-dependent manner due to complementary hydrogen bonding with the corresponding nucleotide in the template fragment.

A polymerase enzyme may subsequently catalyze the chemical addition of the next complementary nucleotide into the nucleic acid chain.

In each sequencing cycle, the next complementary nucleotide is identified by excitation of the fluorophore, or light emitting marker in the disclosed method, and detection of the emitted fluorescence, for example, using laser excitation and imaging.

The nucleotide may comprise a “terminator” which may be a “reversible terminator”. A nucleotide having a terminator or reversible terminator moiety can be used such that subsequent extension cannot occur until a deblocking agent is delivered to remove the terminator moiety. Thus, after nucleotide incorporation and identification, the terminator may be enzymatically cleaved to allow the next sequencing cycle to commence.

The linker attaching the light emitting marker to the nucleotide in the disclosed method may be cleavable or otherwise arranged to allow dissociation of the light emitting marker from the nucleotide. Thus, after incorporation and identification of the test nucleotide, the light emitting marker may be removed to allow the next sequencing cycle to commence and the next complementary nucleotide to be identified. The result is base-by-base sequencing of the template fragment nucleic acids.

The term “linker” is intended to mean a chemical bond or moiety that bridges two moieties, for example by covalent linkage or the formation of a stable complex. A linker can be, for example, the sugar-phosphate backbone that connects nucleotides in a nucleic acid moiety. In the disclosed method, a linker is used to conjugate a test nucleotide to a light emitting marker. The linker can include, for example, one or more of a nucleotide moiety, a nucleic acid moiety, a non-nucleotide chemical moiety, a nucleotide analogue moiety, amino acid moiety, polypeptide moiety, or protein moiety. For example, the linker may comprise a complex comprising a ligand such as biotin and a biomolecule that binds to the ligand with a high affinity, such as avidin, streptavidin, or neutravidin.

The terms “cleave”, “cleavage site”, and similar terms, refer to a moiety in a molecule, such as a linker, that can be modified or removed to physically separate two other moieties of the molecule.

In some embodiments of the disclosed method, a linker may be used to associate the test nucleotide to a terminator. The linker may be a cleavable linker, such that at the appropriate point in the sequencing cycle the linker is cleaved, thereby removing the terminator from the nucleotide. In some embodiments of the disclosed method, the linker that is used to associate the test nucleotide and terminator comprises the same type of cleavable linkage that is present in the linker conjugating the test nucleotide to the light emitting marker. Thus, at the appropriate point in the sequencing cycle, a single agent, such as a single type of cleaving enzyme, may be used to remove both the terminator and light emitting marker from the test nucleotide. In some embodiments of the disclosed method, a single linker may be arranged to conjugate both the terminator and light emitting marker to the test nucleotide. In some embodiments of the disclosed method, the linker that is used to associate the test nucleotide and terminator may comprise a different type of cleavable linkage to that present in the linker conjugating the test nucleotide to the light emitting marker.

In some embodiments of the disclosed method, the linker is a cleavable linker, for example a chemically cleavable linker or a photochemically cleavable linker.

A chemically cleavable linker is a linker that cleaves, e.g. separates into two distinct chemical moieties, due to a change in a chemical composition containing the marked linker. For example, a chemically cleavable linker may cleave due to a change in pH or due to the addition of a cleaving agent to the chemical composition (for example a reducing agent, an oxidising agent, an enzyme and/or a catalyst). Non-limiting examples of such cleaving agents are disclosed in Leriche et al, “Cleavable linkers in chemical biology”, Bioorganic & Medicinal Chemistry, Vol. 20, Issue 2, 2012, p.57 1-582, the contents of which are incorporated herein in its entirety, and include, without limitation, transition metals, thiols (e.g. dithiothreitol), hydrazines, formic acid and/or tris(hydroxypropyl) phosphine).

A photochemically cleavable linker is a linker that separates into two distinct chemical moieties when irradiated. For example, a photochemically cleavable linker may separate when irradiated with light with a peak wavelength in the range of about 200 nm to about 2500 nm, about 200 nm to about 1000 nm, or about 300 nm to about 1000 nm. In one embodiment, a photochemically cleavable linker may separate when irradiated with light with a peak wavelength in the range of about 300 nm to about 500 nm, for example about 340 nm. Exemplary photocleavable linkers are disclosed in Seok Ki Choi “Photocleavable linkers: design and application technology” Chapter 9, Photonanotechnology for Therapeutics and Imaging, Micro and Nano Technologies, 2020, pages 243-275, and Leriche et al, “Cleavable linkers in chemical biology”, Bioorganic & Medicinal Chemistry, Vol. 20, Issue 2, 2012, p.571-582, the contents of which are incorporated herein by reference.

Exemplary cleavable linkers include: disulfides which may be cleaved by, e.g., dithiothreitol and phosphines such as tris-(2-carboxyethyl)phosphine; azo compounds which may be cleaved by, e.g., a dithionite such as sodium dithionite; carbamates which may be cleaved by an acid, e.g., trifluoroacetic acid; silanes which may be cleaved by, e.g., formic acid; hydrazide imines which may be cleaved by, e.g., aceylhydrazine; hydrazine imines which may be cleaved by, e.g., hydroxylamine; nitrobenenzesulfonamide which may be cleaved by, e.g., 2-mercaptoethanol; sulfonamides which may be cleaved by, e.g., hydroxylamine/iodoacetonitrile; and groups that may be cleaved by a transition metal or a phosphine catalyst such as ethers having a disulfide alpha substituent and ethers having an azide alpha substituent. The cleavable linker may associate the test nucleotide with the light-emitting marker (or in alternative methods, a terminator). In some embodiments, the cleavable linker associates the test nucleotide with the light-emitting marker by attaching to either or both of the test nucleotide and the light-emitting marker. For example, the cleavable linker is attached to either or both of the test nucleotide and the light-emitting marker.

In some embodiments, the cleavable linker is attached to the light-emitting polymer of the light-emitting marker.

In some embodiments, when the light-emitting marker is a particulate light-emitting marker, the cleavable linker is attached to the light-emitting material (for example the light-emitting polymer) of the particulate light-emitting marker. In some embodiments, when the light-emitting marker is a particulate light-emitting marker, the cleavable linker is attached to a surface or matrix of the particulate light-emitting marker. In some embodiments, the surface or matrix of the particulate light-emitting marker is functionalised (for example by the grafting of polyethylene glycol chains thereto) such that the cleavable linker can attach to said surface or matrix.

In some embodiments, the cleavable linker comprises a cleavable moiety. The cleavable moiety may be directly attached to either or both of the test nucleotide and the light-emitting marker (or in alternative methods, a terminator). In some embodiments, the cleavable moiety may be attached to one or more intermediate moieties, which may each individually be directly attached to either or both of the test nucleotide and the light-emitting marker (or in alternative methods, a terminator). In some embodiments, the cleavable moiety is attached to either of the ligand or biomolecule. For example, the cleavable linker is attached to the light-emitting marker by a stable complex formed between a ligand and a biomolecule. For example, the cleavable linker is attached to the test nucleotide by a stable complex formed between a ligand and a biomolecule.

In some embodiments, the cleavable linker comprises a cleavable moiety represented by any of Formula 1-6:

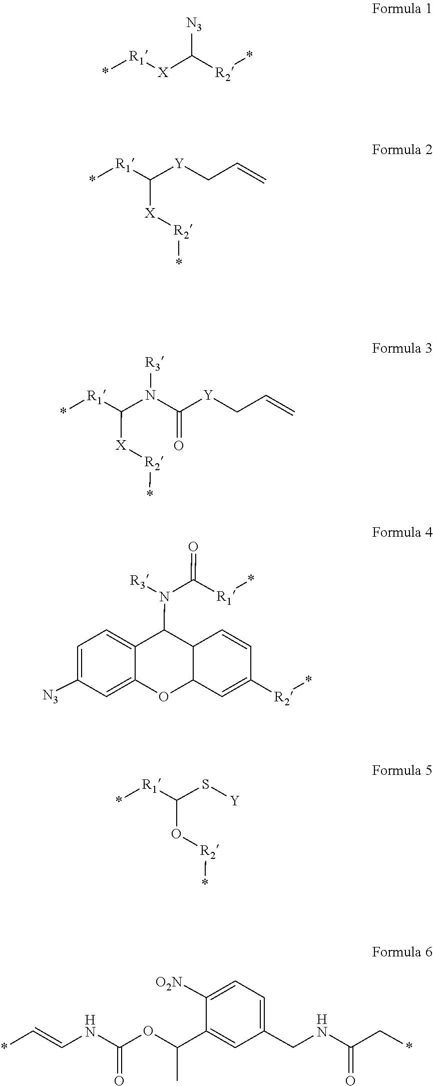

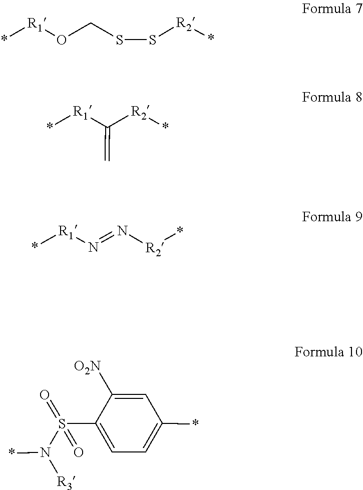

In each of Formulae 1-10:
each X is independently O, S, or NR₃′;
each Y is independently O, S, NH or N(C₁-C₃₀ allyl);
each R₁′ and R₂′ is independently is a direct bond, a substituted or unsubstituted C₁-C₆₀ alkylene group, a substituted or unsubstituted C₂-C₆₀ alkenylene group, a substituted or unsubstituted C₂-C₆₀ alkynylene group, a substituted or unsubstituted C₁-Coo alkoxylene group, a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₆-C₆₀ aryleneoxy group, a substituted or unsubstituted C₆-C₆₀ arylenethio group, a substituted or unsubstituted C₁-C₆₀ heteroarylene group, a substituted or unsubstituted monovalent non-aromatic condensed C8-C60 polycyclic group, or a substituted or unsubstituted monovalent non-aromatic condensed 8 to 60 membered heteropolycyclic group.
each R₃′ is independently hydrogen or a C₁-C₃₀ alkyl group; and
* denotes an attachment to a neighboring atom.

In some embodiments, the neighboring atom is an atom of a test nucleotide, a light-emitting marker (for example, a light-emitting polymer of a light-emitting marker, a light-emitting material of a particulate light-emitting marker, or a surface or matrix of a particulate light-emitting marker), a ligand or a biomolecule.

Optionally, the biomolecule is a protein. Optionally, the ligand is an antigen and the biomolecule comprises an antigen-binding fragment. Optionally, the biomolecule is an antibody. Optionally, the ligand is biotin and the biomolecule is selected from avidin, streptavidin, neutravidin and recombinant variants thereof.

A non-limiting example a cleavable moiety according to Formula 1 is:

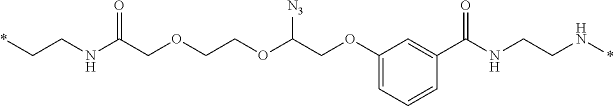 [SMILES: *CCNC(=O)COCCOC(COc1cccc(C(=O)NCCN*)c1)N=[N+]=[N-]]

Non-limiting examples of cleavable moieties according to Formula 7 are:

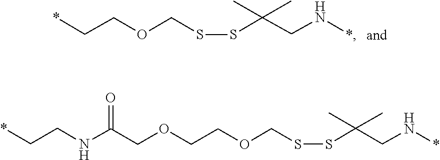

A non-limiting example a cleavable moiety according to Formula 8 is:

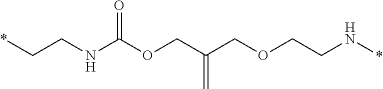 [SMILES: *CCNC(=O)OCC(=C)COCCN*]

A non-limiting example a cleavable moiety according to Formula 9 is:

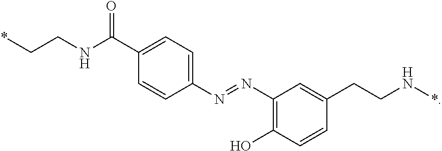

In some embodiments of the disclosed method, the reaction mixture includes a single species of test nucleotide, such as, for example, dATP, dCTP, dGTP, or dTTP only. In these embodiments, the detection of fluorescence emission from the associated light emitting marker after incorporation of the test nucleotide identifies the test nucleotide as the next correct nucleotide in the sequence. In these embodiments, repeated sequencing cycles may be used to test a different species of test nucleotide sequentially.

In some embodiments, a plurality, such as two, three, or four different species of test nucleotide may be included in the reaction mixture in a single sequencing cycle. Each of the different species of test nucleotide may be associated with different light emitting markers. Thus, the light emitting markers associated with each different nucleotide species may be distinguishable and identifiable, for example on the basis of different fluorescence emission spectra. In these embodiments, the fluorescence emission that is detected is used to determine which of the different species of test nucleotides is incorporated into the primed strand and, thus represents the next complementary nucleotide.

In some embodiments, a plurality of different nucleic acid fragments can be sequenced simultaneously under conditions where events occurring for different templates can be distinguished, for example due to being present at different locations in an array.

Current sequencing methods are known to suffer from errors known as pre-phasing and post-phasing. These errors occur as a result of the loss of uniformity in the identity of the fluorescent label incorporated in a cluster. For example, errors may occur due to the non-removal of a fluorescent label, and/or the non-incorporation of a nucleotide. These errors may accumulate with increased numbers of sequencing cycles. In current sequencing methods, the typical error rate is approximately 0.2%. Thus, in each sequencing cycle, errors occur in an average of two templates per cluster of 1000.

As a result of such errors in current sequencing methods, as the number of sequencing cycles increases, the proportion of template fragments in a cluster comprising an incorrect label increases. Eventually, the accumulation of errors is such that it becomes impossible to accurately identify the next nucleotide. Thus, in current methods, this effect imposes a limit on the length of template fragments (i.e. number of nucleotides) that can be sequenced in a single sequencing read. Current sequencing methods are thus limited to read lengths in the order of up to about 300 bp.

An advantage of the disclosed method, however, is that the smaller number of template fragments used in each cluster and the high fluorescence intensity and low signal to noise ratio of the disclosed light emitting markers allows errors to be recognized and taken into account. The use of high brightness light emitting markers provides the advantage of reduced sample sizes allowing identification of individual phase shifting events, and thus compensation of them in data interpretation.

In some embodiments, the disclosed method further provides a method of improved error recognition and/or correction. Due to the use of smaller clusters of cloned template molecules, any reduction in signal arising from an error in the sequencing process, such as the non-removal of a fluorescent label, and/or the non-incorporation of a nucleotide, may be detected as a significant step-change in the total level of fluorescence emitted by a cluster. Thus, in current methods, an error occurring in one template molecule in a cluster of 1000 strands may not provide a detectable change in the total fluorescence emitted by the cluster (i.e. a positive signal from 1000 fluorophores versus a positive signal from 999 fluorophores). Thus, errors slowly accumulate until correct nucleotide detection is impossible. In contrast, in the disclosed method, in the case of a cluster comprising 10 template strands, the change in fluorescence occurring as a result of an error in one strand may be significant and detectable (i.e. a positive signal from 10 light emitting markers versus a positive signal from 9 light emitting markers). This step-change in fluorescence, and the knowledge of the cycle in which it occurred, may be used to inform the data analysis performed in relation to subsequent sequencing cycles.

The disclosed method comprising the use of the disclosed light emitting markers, for example, with brightness in excess of 3×10⁶ M⁻¹cm⁻¹, enables read lengths for sequence by synthesis methods to be extended in comparison to prior art methods. For example, read lengths in excess of 500, in excess of 800 or in excess of 1000 base pairs are possible, as a result of the disclosed method allowing improved error detection and subsequent correction.

4. Data Analysis

During data analysis, the newly identified sequence reads of the template fragment are aligned, and the target nucleic acid sequence may thus be determined.

Following alignment, many variations of analysis are possible, including single nucleotide polymorphism (SNP), insertion-deletion (indel) identification, and read counting for RNA methods, phylogenetic or metagenomic analysis.

### Example Embodiments

FIG. 1 shows an example of a first embodiment of the disclosed method.

In step (I), a primed template nucleic acid fragment 1 is immobilised on a support substrate 2. A reaction mixture comprising DNA polymerase enzyme 3 and test nucleotide 4 are provided. The test nucleotide is conjugated to a biotin moiety 5 by a cleavable linker.

In step (II), the test nucleotide is incorporated into the primed DNA strand by the polymerase enzyme. A light emitting marker 6, comprising a light emitting polymer 7 in a silica matrix 8 is provided. The light emitting marker 6 comprises surface-bound streptavidin 9.

A shown in step (III), a stable complex is formed between the biotin moiety 5 of the test nucleotide 4 and the streptavidin moiety 9 of the light emitting marker 6. The result is a marked nucleotide 10 comprising the test nucleotide conjugated to a light emitting marker. Thus, the marked nucleotide comprising the test nucleotide is formed after incorporation of the test nucleotide into the primed strand.

Detecting the light emitted by the light emitting marker 6 identifies that the test nucleotide 4 is incorporated into the primed strand 1, and thus the test nucleotide comprises a base complementary to the next base of the template strand.

To complete the sequencing cycle, the linker is cleaved 11.

The skilled person will appreciate that various modifications and adaptations may be made to the example embodiment shown in FIG. 1.

For example, in the embodiment of FIG. 1, a combination of biotin on the test nucleotide and streptavidin on the light emitting marker are used to facilitate formation in step (III) of the marked nucleotide. The skilled person will appreciate that the ligand (biotin) and biomolecule (streptavidin) may be used in the opposite orientation, or indeed, any combination of ligand and biomolecule that are capable of forming a stable complex may be used, in either orientation. For example, the ligand may be an antigen and the biomolecule may be an antibody or an antigen-binding fragment of an antibody.

In some embodiments, such as for example, in embodiments where the nucleotide is conjugated to biotin, the reaction mixture used in step (I) may comprise a single species of biotinylated test nucleotide, with the reaction cycle being repeated with a different species of test nucleotide used in each cycle. In this case, in step (II), the same (e.g. streptavidin-conjugated) light emitting marker may be used in each cycle to form the marked nucleotide. This is because detection of fluorescence emission from the light emitting marker simply identifies whether or not the test nucleotide is the correct next nucleotide.

In other embodiments, the detection of fluorescence emission from the light emitting marker may be used to identify which of a plurality of different test nucleotides comprises a base that is complementary to the next base of the template strand. For example, in embodiments where the nucleotide is conjugated to an antigen, the reaction mixture used in step (I) may comprise a plurality of different species of test nucleotides, each of the different species being conjugated to a different antigen. In this case, for detection in step (II), a different antibody-conjugated light emitting marker may be used, for example, arranged such that each light emitting marker can be used to identify a different test nucleotide, and thereby identify the test nucleotide that is incorporated into the primed strand, and hence comprises a base that is complementary to the next base of the template strand.

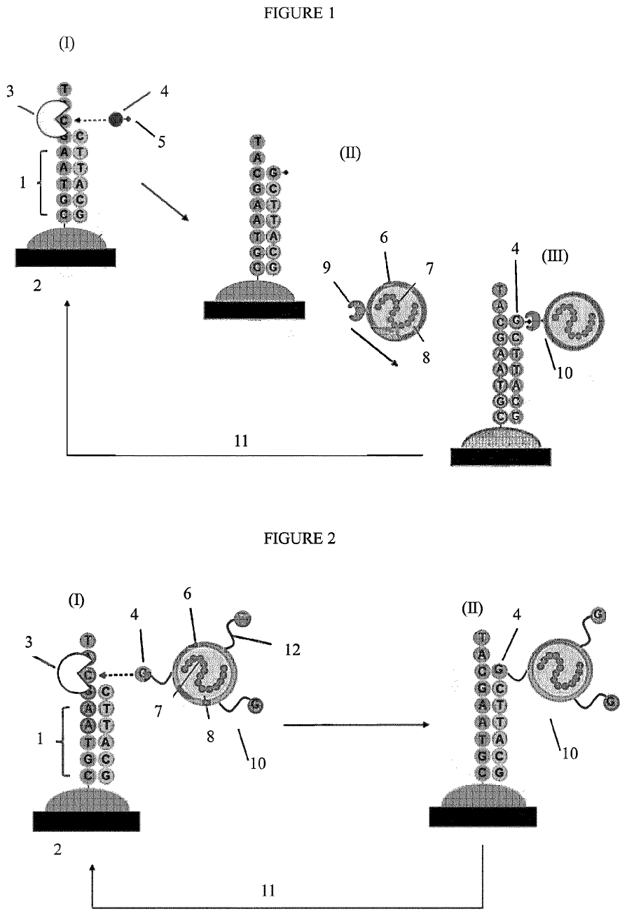

FIG. 2 shows an example of a second embodiment of the disclosed method.

In step (I), a primed template nucleic acid fragment 1 is immobilised on a support substrate 2. The fragment 1 is contacted with a reaction mixture comprising DNA polymerase enzyme 3 and a marked nucleotide 10. The marked nucleotide 10 comprises a test nucleotide 4 conjugated to a light emitting marker 6 by a linker in the form of a flexible spacer 12. The light emitting marker 6 comprises a light emitting polymer 7 in a matrix 8. Thus, in this embodiment, the test nucleotide is present in the reaction mixture in the form of a marked nucleotide.

In step (II), the test nucleotide is incorporated into the primed DNA strand by the polymerase enzyme. Detecting the light emitted by the light emitting marker 6 identifies that the test nucleotide 4 is incorporated into the primed strand 1, and thus identifies that the test nucleotide comprises a base complementary to the next base of the template strand.

To complete the sequencing cycle, the linker is cleaved 11.

Other Methods

The skilled person will appreciate that the disclosed marked nucleotides may be used in any method involving the use of fluorescence to identify a nucleotide. Such methods include, for example, methods for the analysis of short tandem repeat (STR) markers, single nucleotide polymorphisms (SNPs), methylation patterns, ChIP analysis, and RNA transcription.

It also includes methods of analysis of any nucleic acid template, including, for example, DNA from any organism or mixed population such as a microbiome, whole genomes, RNA transcripts for expression analysis, cancer samples (such as methods of analysing somatic variants and/or tumour subclones), and mitochondrial DNA.

Light Emitting Marker

In some embodiments, the light emitting marker comprises or consists of a light-emitting polymer. In use, the light-emitting polymer may be dissolved or dispersed in the reaction mixture.

In some embodiments, the light-emitting marker is a particulate light-emitting marker comprising a light-emitting material.

The light-emitting material of the light-emitting marker may emit light having a peak wavelength in the range of 350-1000 nm.

A blue light-emitting material as described herein may have a photoluminescence spectrum with a peak of no more than 500 nm, preferably in the range of 400-500 nm, optionally 400-490 nm.

A green light-emitting material as described herein may have a photoluminescence spectrum with a peak of more than 500 nm up to 580 nm, optionally more than 500 nm up to 540 nm.

A red light-emitting material as described herein may have a photoluminescence spectrum with a peak of no more than more than 580 nm up to 950 nm, optionally up to 630 nm, optionally 585 nm up to 625 nm.

The light-emitting material may have a shift between excitation and emission maxima in the range of 20-400 nm.

UV/vis absorption spectra of light-emitting markers as described herein may be as measured in methanol solution or suspension using a Cary 5000 UV-vis-IR spectrometer.

Photoluminescence spectra of light-emitting particles as described herein may be measured in methanol solution or suspension using a Jobin Yvon Horiba Fluoromax-3.

Light-Emitting Polymer

A light-emitting polymer as described herein may be a fluorescent or phosphorescent light-emitting polymer. In use, the light-emitting polymer may be dissolved in the reaction mixture or a particulate light-emitting marker comprising or consisting of the light-emitting polymer may be dispersed in the reaction mixture.

The light-emitting polymer may be a homopolymer or may be a copolymer comprising two or more different repeat units.

The light-emitting polymer may comprise light-emitting groups in the polymer backbone, pendant from the polymer backbone or as end groups of the polymer backbone. In the case of a phosphorescent polymer, a phosphorescent metal complex, preferably a phosphorescent iridium complex, may be provided in the polymer backbone, pendant from the polymer backbone or as an end group of the polymer backbone.

The light-emitting polymer may have a non-conjugated backbone or may be a pi-conjugated polymer. By “pi-conjugated polymer” is meant a polymer comprising repeat units in the polymer backbone that are directly pi-conjugated to adjacent repeat units. Pi-conjugated light-emitting polymers include, without limitation, polymers comprising one or more of arylene, heteroarylene and vinylene groups pi-conjugated to one another along the polymer backbone.

The light-emitting polymer may have a linear, branched or crosslinked backbone.

The light-emitting polymer may comprise one or more repeat units in the backbone of the polymer substituted with one or more substituents selected from non-polar and polar substituents.

Preferably, the light-emitting polymer comprises at least one polar substituent. The one or more polar substituents may be the only substituents of said repeat units, or said repeat units may be further substituted with one or more non-polar substituents, optionally one or more C₁₋₄₀ hydrocarbyl groups. The repeat unit or repeat units substituted with one or more polar substituents may be the only repeat units of the polymer or the polymer may comprise one or more further co-repeat units wherein the or each co-repeat unit is unsubstituted or is substituted with non-polar substituents, optionally one or more C₁₋₄₀ hydrocarbyl substituents.

C₁₋₄₀ hydrocarbyl substituents as described herein include, without limitation, C₁₋₂₀ alkyl, unsubstituted phenyl and phenyl substituted with one or more C₁₋₂₀ alkyl groups.

As used herein a “polar substituent” may refer to a substituent, alone or in combination with one or more further polar substituents, which renders the light-emitting polymer with a solubility of at least 0.01 mg/ml in an alcoholic solvent, optionally in the range of 0.01-10 mg/ml. Optionally, solubility is at least 0.1 or 1 mg/ml. The solubility is measured at 25° C. Preferably, the alcoholic solvent is a C₁₋₁₀ alcohol, more preferably methanol.

Polar substituents are preferably substituents capable of forming hydrogen bonds or ionic groups.

In some embodiments, the light-emitting polymer comprises polar substituents of formula —O(R³O)ₜ—R⁴ wherein R³ in each occurrence is a C₁₋₁₀ alkylene group, optionally a C₁₋₅ alkylene group, wherein one or more non-adjacent, non-terminal C atoms of the alkylene group may be replaced with O, R⁴ is H or C₁₋₅ alkyl, and t is at least 1, optionally 1-10. Preferably, t is at least 2. More preferably, t is 2 to 5. The value of t may be the same in all the polar groups of formula —O(R³O)ₜ—R⁴. The value of t may differ between polar groups of the same polymer.

By “C₁₋₅ alkylene group” as used herein with respect to R³ is meant a group of formula —(CH₂)_{f}— wherein f is from 1-5.

Preferably, the light-emitting polymer comprises polar substituents of formula —O(CH₂CH₂O)ₜ—R⁴ wherein t is at least 1, optionally 1-10 and R⁴ is a C₁₋₅ alkyl group, preferably methyl. Preferably, t is at least 2. More preferably, t is 2 to 5, most preferably t is 3.

In some embodiments, the light-emitting polymer comprises polar substituents of formula —N(R⁵)₂, wherein R⁵ is H or C₁₋₁₂hydrocarbyl. Preferably, each R⁵ is a C₁₋₁₂ hydrocarbyl.

In some embodiments, the light-emitting polymer comprises polar substituents which are ionic groups which may be anionic, cationic or zwitterionic. Preferably the ionic group is an anionic group.

Exemplary anionic groups are —COO⁻, a sulfonate group; hydroxide; sulfate; phosphate; phosphinate; or phosphonate.

An exemplary cationic group is —N(R⁵)₃_{⁺} wherein R⁵ in each occurrence is H or C₁₋₁₂ hydrocarbyl. Preferably, each R⁵ is a C₁₋₁₂hydrocarbyl.

A light-emitting polymer comprising cationic or anionic groups comprises counterions to balance the charge of these ionic groups.

An anionic or cationic group and counterion may have the same valency, with a counterion balancing the charge of each anionic or cationic group.

The anionic or cationic group may be monovalent or polyvalent. Preferably, the anionic and cationic groups are monovalent.

The light-emitting polymer may comprise a plurality of anionic or cationic polar substituents wherein the charge of two or more anionic or cationic groups is balanced by a single counterion. Optionally, the polar substituents comprise anionic or cationic groups comprising di- or trivalent counterions.

The counterion is optionally a cation, optionally a metal cation, optionally Li⁺, Na⁺, K⁺, Cs⁺, preferably Cs⁺, or an organic cation, optionally ammonium, such as tetraalkylammonium, ethylmethyl imidazolium or pyridinium.

The counterion is optionally an anion, optionally a halide; a sulfonate group, optionally mesylate or tosylate; hydroxide; carboxylate; sulfate; phosphate; phosphinate; phosphonate; or borate.

In some embodiments, the light-emitting polymer comprises polar substituents selected from groups of formula —O(R³O)ₜ—R⁴, groups of formula —N(R⁵)₂, groups of formula OR⁴ and/or ionic groups. Preferably, the light-emitting polymer comprises polar substituents selected from groups of formula —O(CH₂CH₂O)ₜR⁴, groups of formula —N(R⁵)₂, and/or anionic groups of formula —COO⁻. Preferably, the polar substituents are selected from the group consisting of groups of formula —O(R³O)ₜ—R⁴, groups of formula —N(R⁵)₂, and/or ionic groups. Preferably, the polar substituents are selected from the group consisting of polyethylene glycol (PEG) groups of formula —O(CH₂CH₂O)ₜR⁴, groups of formula —N(R⁵)₂, and/or anionic groups of formula —COO⁻, R³, R⁴, R⁵, and t are as described above.

Optionally, the backbone of the light-emitting polymer is a pi-conjugated polymer.

Optionally, the backbone of the pi-conjugated light-emitting polymer comprises repeat units of formula (III):

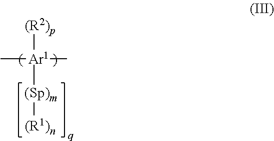

wherein Ar¹ is an arylene group or heteroarylene group; Sp is a spacer group; m is 0 or 1; R¹ independently in each occurrence is a polar substituent; n is 1 if m is 0 and n is at least 1, optionally 1, 2, 3 or 4, if m is 1; R² independently in each occurrence is a non-polar substituent; p is 0 or a positive integer, optionally 1, 2, 3 or 4; q is 0 or a positive integer, optionally 1, 2, 3 or 4; and wherein Sp, R¹ and R² may independently in each occurrence be the same or different.

Preferably, m is 1 and n is 2-4, more preferably 4. Preferably p is 0.

Ar¹ of formula (III) is optionally a C₆₋₂₀ arylene group or a 5-20 membered heteroarylene group. Ar¹ is preferably a C₆₋₂₀ arylene group or silafluorene, optionally phenylene, fluorene, benzofluorene, phenanthrene, naphthalene or anthracene, more preferably fluorene or phenylene, most preferably fluorene.

Sp-(R¹)n may be a branched group, optionally a dendritic group, substituted with polar groups, optionally —NH₂ or —OH groups, for example polyethyleneimine.

Preferably, Sp is selected from:
C₁₋₂₀ alkylene or phenylene-C₁₋₂₀ alkylene wherein one or more non-adjacent C atoms may be replaced with O, S, N or C═O;
a C₆₋₂₀ arylene or 5-20 membered heteroarylene, more preferably phenylene, which, in addition to the one or more substituents R¹, may be unsubstituted or substituted with one or more non-polar substituents, optionally one or more C₁₋₂₀ alkyl groups.

“alkylene” as used herein means a branched or linear divalent alkyl chain.

“non-terminal C atom” of an alkyl group as used herein means a C atom other than the methyl group at the end of an n-alkyl group or the methyl groups at the ends of a branched alkyl chain.

More preferably, Sp is selected from:
C₁₋₂₀ alkylene wherein one or more non-adjacent C atoms may be replaced with O, S or CO; and
a C₆₋₂₀ arylene or a 5-20 membered heteroarylene, even more preferably phenylene, which may be unsubstituted or substituted with one or more non-polar substituents.

R¹ may be a polar substituent as described anywhere herein. Preferably, R¹ is:
a polyethylene glycol (PEG) group of formula —O(CH₂CH₂O)ₜR⁴ wherein t is at least 1, optionally 1-10 and R⁴ is a C₁₋₅ alkyl group, preferably methyl;
a group of formula —N(R⁵)₂, wherein R⁵ is H or C₁₋₁₂ hydrocarbyl; or
an anionic group of formula —COO⁻.

In the case where n is at least two, each R¹ may independently in each occurrence be the same or different. Preferably, each R¹ attached to a given Sp group is different.

In the case where p is a positive integer, optionally 1, 2, 3 or 4, the group R² may be selected from:
alkyl, optionally C₁₋₂₀ alkyl; and
aryl and heteroaryl groups that may be unsubstituted or substituted with one or more substituents, preferably phenyl substituted with one or more C₁₋₂₀ alkyl groups;
a linear or branched chain of aryl or heteroaryl groups, each of which groups may independently be substituted, for example a group of formula —(Ar³)ₛ wherein each Ar³ is independently an aryl or heteroaryl group and s is at least 2, preferably a branched or linear chain of phenyl groups each of which may be unsubstituted or substituted with one or more C₁₋₂₀ alkyl groups; and
a crosslinkable-group, for example a group comprising a double bond such and a vinyl or acrylate group, or a benzocyclobutane group.

Preferably, each R², where present, is independently selected from C₁₋₄₀ hydrocarbyl, and is more preferably selected from C₁₋₂₀ alkyl; unusubstituted phenyl; phenyl substituted with one or more C₁₋₂₀ alkyl groups; and a linear or branched chain of phenyl groups, wherein each phenyl may be unsubstituted or substituted with one or more substituents.

A polymer as described herein may comprise or consist of only one form of the repeating unit of formula (III) or may comprise or consist of two or more different repeat units of formula (III).

Optionally, the polymer comprising one or more repeat units of formula (III) is a copolymer comprising one or more co-repeat units.

If co-repeat units are present then the repeat units of formula (III) may form between 0.1-99 mol % of the repeat units of the polymer, optionally 50-99 mol % or 80-99 mol %. Preferably, the repeat units of formula (III) form at least 50 mol % of the repeat units of the polymer, more preferably at least 60, 70, 80, 90, 95, 98 or 99 mol %. Most preferably the repeat units of the polymer consist of one or more repeat units of formula (III).

The or each repeat unit of the polymer may be selected to produce a desired colour of emission of the polymer.

Arylene repeat units of the polymer include, without limitation, fluorene, preferably a 2,7-linked fluorene; phenylene, preferably a 1,4-linked phenylene; naphthalene, anthracene, indenofluorene, phenanthrene and dihydrophenanthrene repeat units.

The polystyrene-equivalent number-average molecular weight (Mn) measured by gel permeation chromatography of the light-emitting polymers described herein may be in the range of about 1×10³ to 1×10⁸, and preferably 1×10⁴ to 5×10⁶. The polystyrene-equivalent weight-average molecular weight (Mw) of the polymers described herein may be 1×10³ to 1×10⁸, and preferably 1×10⁴ to 1×10⁷.

Polymers as described herein are suitably amorphous polymers.

Particulate Light-Emitting Marker

A particulate light-emitting marker as described herein may be, without limitation, a micro- or nano-particulate light-emitting marker.

In some embodiments, the particulate light emitting marker comprises or consists of a quantum dot. Exemplary light-emitting quantum dot materials include, without limitation, metal chalcogenides. Quantum dots include, without limitation, core, core-shell and alloyed quantum dots.

In some embodiments, the particulate light-emitting marker is a collapsed light-emitting polymer.

In some embodiments, the light-emitting particles of the particulate light-emitting marker comprise a light-emitting material and a matrix. The light-emitting material may be a fluorescent or phosphorescent light-emitting material. The light-emitting material be polymer or non-polymeric.

Exemplary non-polymeric fluorescent materials include, without limitation: fluorescein, fluorescein isothiocyanate (FITC); fluorescein NHS; Alexa Fluor 488; Dylight 488; Oregon green; DAF-FM; 6-FAM; 2,7-dichlorofluorescein; 3′-(p-aminophenyl)fluorescein; 3′-(hydroxyphenyl)fluorescein; rhodamines, for example Rhodamine 6G and Rhodamine 110 chloride; coumarins; boron-dipyrromethenes (BODIPYs); naphthalimides; perylenes; benzanthrones; benzoxanthrones; benzothiooxanthrones; 2-(4-pyridyl)-5-phenyl-oxazole; 2-quinolinyl-5-phenyl-oxazole; 2-(4-pyridyl)-5-naphthyl-oxazole; 2-(4-pyridyl)-5-phenyl-thiazole; 2-quinolinyl-5-phenyl-thiazole; 2-(4-pyridyl)-5-naphthyl-thiazole; 2-(4-pyridyl)-5-phenyl-thiophene; 2-quinolinyl-5-phenyl-thiophene; 2-(4-pyridyl)-5-naphthyl-thiophene and salts thereof, each of which may be unsubstituted or substituted with one or more substituents. Exemplary substituents are ionic or non-ionic substituents as described herein, optionally chlorine, alkyl amino; phenylamino; and hydroxyphenyl.

In the case where a particulate light-emitting marker as described herein comprises a light-emitting polymer, the light-emitting polymer is optionally selected from light-emitting polymers described above.

Preferably, the light-emitting particles as described herein have a number average diameter of no more than 500 nm or 400 nm in methanol as measured by dynamic light scattering (DLS) using a Malvern Zetasizer Nano ZS (Details of measurement in the Examples). Preferably the particles have a number average diameter of between 5-500 nm, optionally 10-200 nm, preferably between 20-150 nm, as measured by a Malvern Zetasizer Nano ZS. The inventors have found that light emitting particles having an average diameter of less than 50 nm, such as 20-40 nm are preferred. In addition, the inventors have produced light emitting particles having a diameter of 30 nm which have an extinction coefficient that is at least two orders of magnitude higher than that of a typical small molecule dye. Particles of this size are also ideally suited for use in current sequencing methods, for example, using substrate beads having diameters in the range of 1-2 μm, or nanowells in the range of 200-600 nm.

Matrix-Containing Light-Emitting Particles

The matrix of a light-emitting particle comprising a matrix and a light-emitting material may at least partially isolate the light-emitting material from the surrounding environment. This may limit any effect that the external environment may have on the lifetime of the light-emitting material.

In some embodiments, the particle comprises the light-emitting material homogenously distributed through the matrix.

In some embodiments, the particle may have a particulate core and, optionally, a shell wherein at least one of the core and shell contains the light-emitting material.

In the case where the light-emitting material is a polymer, polymer chains of the light-emitting polymer may extend across some or all of the thickness of the core and/or shell. Polymer chains may be contained within the core and/or shell or may protrude through the surface of the core and/or shell.

In some embodiments, the particle comprises a core comprising or consisting of the light-emitting polymer and a shell comprising or consisting of the matrix.

In some embodiments, the particle core consists of the matrix and the light-emitting material. In some embodiments, the particle core comprises at least one further material, for example a host material configured to absorb excitation energy from an energy source, e.g. a light source, and transfer energy to the transferring energy to the light-emitting material.

Exemplary polymeric matrix materials include, without limitation, polystyrene and homopolymers or copolymers of (alkyl)acrylic acids. A polymeric matrix material may be crosslinked, e.g. a crosslinked chitosan-polyacrylic acid polymer. The polymer matrix may be a self-assembled micelle or vesicle comprising lipid or polymer surfactants. The polymer matrix is preferably an inorganic oxide, optionally silica, alumina or titanium dioxide. The polymer matrix is more preferably silica.

In some embodiments the light-emitting material may be covalently bound, directly or indirectly, to the matrix material. In some embodiments, the light-emitting material may be mixed with (i.e. not covalently bound to) a matrix material. Preferably, the matrix is not covalently bound to the light-emitting material, in which case there is no need for the matrix material and/or the light-emitting material to be substituted with reactive groups for forming such covalent bonds, e.g. during formation of the particles.

In some embodiments, a silica matrix as described herein may be formed by polymerisation of a silica monomer in the presence of the light-emitting material, for example as described in WO 2018/060722, the contents of which are incorporated herein by reference.

In some embodiments, the polymerisation comprises bringing a solution of silica monomer into contact with an acid or a base. The acid or base may be in solution. The light-emitting material may be in solution with the acid or base and/or the silica monomer before the solutions are mixed. Optionally, the solvents of the solutions are selected from water, one or more C₁₋₈ alcohols or a combination thereof.

Polymerising a matrix monomer in the presence of a light-emitting polymer may result in one or more chains of the polymer encapsulated within the particle and/or one or more chains of the polymer extending through a particle.

The particles may be formed in a one-step polymerisation process.

Optionally, the silica monomer is an alkoxysilane, preferably a trialkoxy or tetra-alkoxysilane, optionally a C₁₋₁₂ trialkoxy or tetra-alkoxysilane, for example tetraethyl orthosilicate. The silica monomer may be substituted only with alkoxy groups or may be substituted with one or more groups.

Optionally, at least 0.1 wt % of total weight of the particle core consists of the light-emitting material. Preferably at least 1, 10, 25 wt % of the total weight of the particle core consists of the light-emitting material.

Optionally at least 50 wt % of the total weight of the particle core consists of the matrix. Preferably at least 60, 70, 80, 90, 95, 98, 99, 99.5, 99.9 wt % of the total weight of the particle core consists of the matrix.

### EXAMPLES

### Example 1

### Materials Preparation

Buffer Preparation

Borate Buffer

Sodium tetraborate solution (aq., 50 mM), was added slowly to a stirred solution of boric acid (aq., 50 mM) until the solution reached pH 8.3.

Citrate Buffer

To a stirred solution of sodium citrate dihydrate (13.405 g, 45.6 mmol) and citric acid (10.454 g, 54.4 mmol) in deionised water (400 ml), sodium hydroxide (2M) was added portion wise until pH 4.5 was reached. The resulting solution was diluted (with stirring) to 500 ml using deionised water to give a 0.2M solution of sodium citrate buffer.

Solution Preparation

1% BSA Solutions

1 wt % bovine serum albumin (BSA) solutions of borate buffer and citrate buffer were prepared by dissolving BSA (300 mg) in the appropriate buffer (30 ml), using a vortex mixer.

50 μg/Ml BSA/Citrate Buffer Solution

60 μl of 1 wt % BSA/sodium citrate buffer was added to 11940 μl of sodium citrate buffer and the resulting mixture was vortex mixed for 5 seconds, to give 12 ml of 50 μg/ml BSA/sodium citrate buffer solution.

50 μg/Ml Biotin-BSA Solution

Lyophilised biotinylated bovine serum albumin (Biotin-BSA) was rehydrated in phosphate buffered saline (PBS) to make a 2 mg/ml stock solution according to the supplier's instructions. 125 μl of the rehydrated biotin-BSA solution was added to 4875 μl of sodium citrate buffer and vortex mixed for 5 seconds to give 5 ml of 50 μg/ml biotin-BSA/sodium citrate buffer solution.

Nanoparticle Synthesis

A nanoparticle (Particle 1) was synthesised by polymerisation of tetraethylorthosilicate (TEOS) in a methanol solution with a polymer of formulation Monomer 1 50 mol %, Monomer 2 40 mol %, Monomer 3 10 mol % dissolved therein, as described in GB 2554666. The reaction was initiated with a ratio of 1 mg of polymer to 100 μL of tetraethylorthosilicate, with a subsequent addition of a further 60 μL of tetraethylorthosilicate after 1 hour. After a further 1 hour the mixture was purified in 3.5 mL portions through 10 mL Zeba desalting columns.

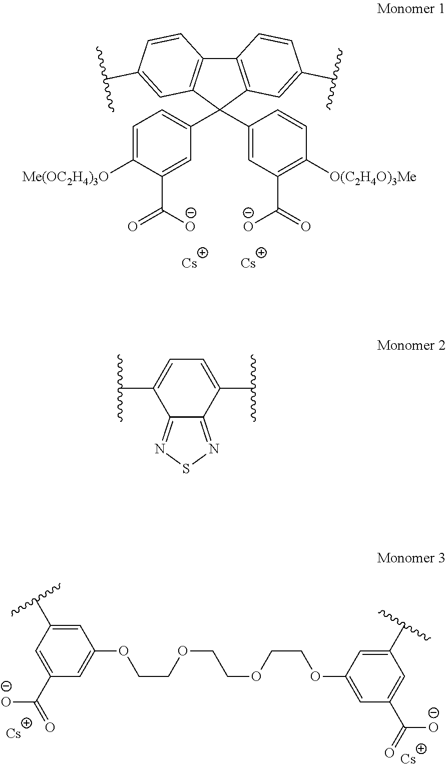

Measurement of Nanoparticle Size

DLS measurements were performed with a Malvern Zetaziser Nano ZS, using a 4 mW 633 nm He—Ne laser. Nanoparticle (Particle 1) suspensions in methanol were tested in single use UV-transparent plastic cuvettes. The machine was operated in Backscatter mode at an angle of 173°. Samples were equilibrated to 25° C. for 60 seconds prior to measurement. Values for the methanol solvent input into the software were 0.5476 cP for viscosity and 1.326 for the refractive index. The sample was defined as Polystyrene latex (RI: 1.590, Absorption: 0.0100). The automatic measurement duration setting was used, with automatic measurement positioning and automatic attenuation. The ‘general purpose’ analysis model was used, with the default size analysis parameters along with a refractive index of 1.59 for the sample parameter. A single measurement was taken for each sample.

Measurement of Extinction Coefficient

A molar extinction coefficient for the fluorescent tags in suspension was measured as follows. A relative molar mass for the nanoparticles (Particle 1) was calculated using the N-ave diameter as measured by Dynamic Light Scattering, described elsewhere in this document, using the assumptions that the particle is spherical and that the density is 1 g/cm³.

The solid content of the suspension was measured by taking the mean of three measurements obtained by gentle evaporation of the liquid from a 200 μL aliquot and weighing of the residue on a balance accurate to 0.1 mg.

The UV/VIS absorption of the suspension was measured at the absorption maximum. A cuvette was filled with 3000 μL of methanol. A series of measurements was taken with 30, 60, 90, 120, 150 μL of the nanoparticles suspension added to the cuvette. The absorption data series was plotted against concentration in mol·L-1 and the extinction coefficient taken as the gradient of the resulting line.

Measurement of Photoluminescence Quantum Yield

The photoluminescence quantum yield (PLQY) of the particles was measured using a Hamamatsu C9920-02 quantum yield spectrometer, with the sample diluted to achieve an absorption of 0.25-0.35 at the peak excitation wavelength.

| Particle | Extinction coefficient | PLQY in H₂O |
|---|---|---|
| Particle 1 | 1 × 10⁷ cm⁻¹M⁻¹ | 0.32 |

Nanoparticle Surface Passivation and Functionalisation

The nanoparticles (Particle 1) were surface functionalised using 39.2 mg mPEG-Silane, MW 1 k and 0.8 mg Biotin-PEG-Silane, MW 1 k to 2 mg of nanoparticles in 1 mL of water. The mixture was stirred at 60° C. for 15½ hours. After cooling the nanoparticles were isolated by pelleting using a centrifuge, removal of the supernatant by pipette and resuspension in water using sonication.

Nanoparticle Functionalisation

709 μl of nanoparticles (Particle 1) in 1 mL water (effective nanoparticle (NP) content: 1.41 mg/ml) was pelleted using the centrifuge (14 k rpm, 20 min), and the supernatant carefully removed using a pipette.

The resulting solid particle was resuspended in 1% BSA/borate buffer (1 ml) using a combination of vortex mixing and ultrasonication. Once the pellet had fully resuspended, a 7 mg/ml solution of streptavidin (350 μg) in borate buffer (50 μl) was added, and the resulting mixture was stirred together for 1 h.

The resulting streptavidin-attached nanoparticle was then pelleted using a centrifuge (14 k rpm, 10 min), the supernatant removed carefully using a pipette and the pellet was then resuspended in 1% BSA/borate buffer (100 μl; for repeat experiments a mixture of 1% BSA/borate buffer, 97 μl, and 0.1 M sodium azide solution, 3 μl, was used) using a combination of vortex mixing and ultrasonication.

The resultant particle suspension of Particle 1 (10 mg/ml in 1% BSA/borate buffer), was analysed by dynamic light scattering (DLS) (Z avg: 78.06, I avg: 90.81, N avg: 49.22, PDI: 0.136) to confirm that the particle was not aggregated.

54 μl of the suspension was diluted into 2106 μl of 1% BSA/borate buffer, to give a suspension of 2160 μl of Particle 1 at 0.25 mg/ml.

FITC Preparation.

FITC-streptavidin (120 μg) was dissolved in 1% BSA/borate buffer (3 ml) to give a 40 μg/ml solution.

Assay Experiment

A 96-well assay plate (Greiner, polystyrene, high & medium binding, flat bottom p-clear plates black sided) was functionalised by the following procedure. An example layout for the assay plate is shown in Table 1 below.

80 μl of 50 μg/ml biotin-BSA was pipetted into each of the wells in column 1, rows A-F of the assay plate.

The 100% biotin-BSA solution was diluted 1:2 into 50 μg/ml BSA/citrate buffer solution and vortex mixed for 5 seconds, to give a combined 50 μg/ml solution of 33% biotin-BSA/66% BSA. 80 μl of this 33% biotin-BSA/BSA solution was pipetted into each of the wells in column 2, rows A-F of the assay plate.

The 33% biotin-BSA/BSA solution was diluted 1:2 into 50 μg/ml BSA/citrate buffer solution and vortex mixed for 5 s, to give a 50 μg/ml solution of 11% biotin-BSA/89% BSA. 80 μl of this 11% biotin-BSA/BSA solution was pipetted into each of the wells in column 3, rows A-F of the assay plate.

This serial dilution procedure was followed to provide the following biotin-BSA/BSA percentages (indicated as B+ in Table 1):
column 1, rows A-F: 100% biotin-BSA/0% BSA (i.e. 50 μg/ml biotin-BSA);
column 2, rows A-F: 33% biotin-BSA/66% BSA (i.e. 16.67 μg/ml biotin-BSA/33.3 μg/ml BSA);
column 3, rows A-F: 11% biotin-BSA/89% BSA (i.e. 5.56 μg/ml biotin-BSA/44.4 μg/ml BSA);
column 4, rows A-F: 3.7% biotin-BSA/96.3% BSA (i.e. 1.85 μg/ml biotin-BSA/48.15 μg/ml BSA);
column 5, rows A-F: 1.2% biotin-BSA/98.8% BSA (i.e. 0.62 μg/ml biotin-BSA/49.38 μg/ml BSA);
column 6, rows A-F: 0.41% biotin-BSA/99.59% BSA (i.e. 0.206 μg/ml biotin-BSA/49.8 μg/ml BSA);
column 7, rows A-F: 0.14% biotin-BSA/99.86% BSA (i.e. 0.069 μg/ml biotin-BSA/49.93 μg/ml BSA);
column 8, rows A-F: 0.046% biotin-BSA/99.95% BSA (i.e. 0.023 μg/ml biotin-BSA/49.97 μg/ml BSA);
column 9, rows A-F: 0.015% biotin-BSA/99.985% BSA (i.e. 0.008 μg/ml biotin-BSA/49.99 μg/ml BSA);
column 10, rows A-F: 5.1×10-3% biotin-BSA/99.995% BSA (i.e. 0.003 μg/ml biotin-BSA/49.99 μg/ml BSA);
column 11, rows A-F: 1.7×10-3% biotin-BSA/99.998% BSA (i.e. 0.001 μg/ml biotin-BSA/49.99 μg/ml BSA);
column 12, rows A-F: 5.6×10-4% biotin-BSA/99.999% BSA (i.e. 0.0003 μg/ml biotin-BSA/49.99 μg/ml BSA).

1% BSA/sodium citrate buffer was added to wells G1-G4, G6-G9, H1-H4, and H6-H9 (indicated as B− in Table 1).

The assay plate was covered and left for 1 h. Wells in rows A-F were emptied, then 1% BSA/sodium citrate buffer (80 μl) was added and the assay plate was recovered and left for a further 1 h.

Wells in rows A-G were then emptied and washed with sodium citrate buffer (80 μl×3 per well).

60 μl of the 0.25 mg/ml solution of nanoparticles (Particle 1; NP) in 1% BSA/borate buffer was added to all wells in rows A-C, and wells G1-G4 (indicated as N in Table 1).

60 μl of the 40 μg/ml solution of FITC-streptavidin in 1% BSA/borate buffer was added to all wells in rows D-F, and wells G6-G9 (indicated as F in Table 1).

The assay plate was covered and left for a further 30 min.

All wells on the assay plate were emptied and wells in rows A-G were washed with borate buffer (80 μl×3 per well) and the dry plate was sealed with sealing tape.

The plate was then measured on the plate reader (NPs: excitation: 473-7.5 nm, emission: 570-20 nm; FITC: excitation: 473-7.5 nm, emission: 530-20 nm).

An example layout for the assay plate is shown in Table 1 below.

TABLE 1

| | % functionalised (biotinylated) surface | | | | | | | | | | | |
|---|---|---|---|---|---|---|---|---|---|---|---|---|
| | 100 | 33 | 11 | 3.7 | 1.2 | 0.41 | 0.14 | 0.046 | 0.015 | 5.1 × 10⁻³ | 1.7 × 10⁻³ | 5.6 × 10⁻⁴ |
| | Column | | | | | | | | | | | |
| Row | 1 | 2 | 3 | 4 | 5 | 6 | 7 | 8 | 9 | 10 | 11 | 12 |
| A | B+, N | B+, N | B+, N | B+, N | B+, N | B+, N | B+, N | B+, N | B+, N | B+, N | B+, N | B+, N |
| B | B+, N | B+, N | B+, N | B+, N | B+, N | B+, N | B+, N | B+, N | B+, N | B+, N | B+, N | B+, N |
| C | B+, N | B+, N | B+, N | B+, N | B+, N | B+, N | B+, N | B+, N | B+, N | B+, N | B+, N | B+, N |
| D | B+, F | B+, F | B+, F | B+, F | B+, F | B+, F | B+, F | B+, F | B+, F | B+, F | B+, F | B+, F |
| E | B+, F | B+, F | B+, F | B+, F | B+, F | B+, F | B+, F | B+, F | B+, F | B+, F | B+, F | B+, F |
| F | B+, F | B+, F | B+, F | B+, F | B+, F | B+, F | B+, F | B+, F | B+, F | B+, F | B+, F | B+, F |
| G | B−, N | B−, N | B−, N | B−, N | | B−, F | B−, F | B−, F | B−, F | | | |
| H | B− | B− | B− | B− | | B− | B− | B− | B− | | | |

Results

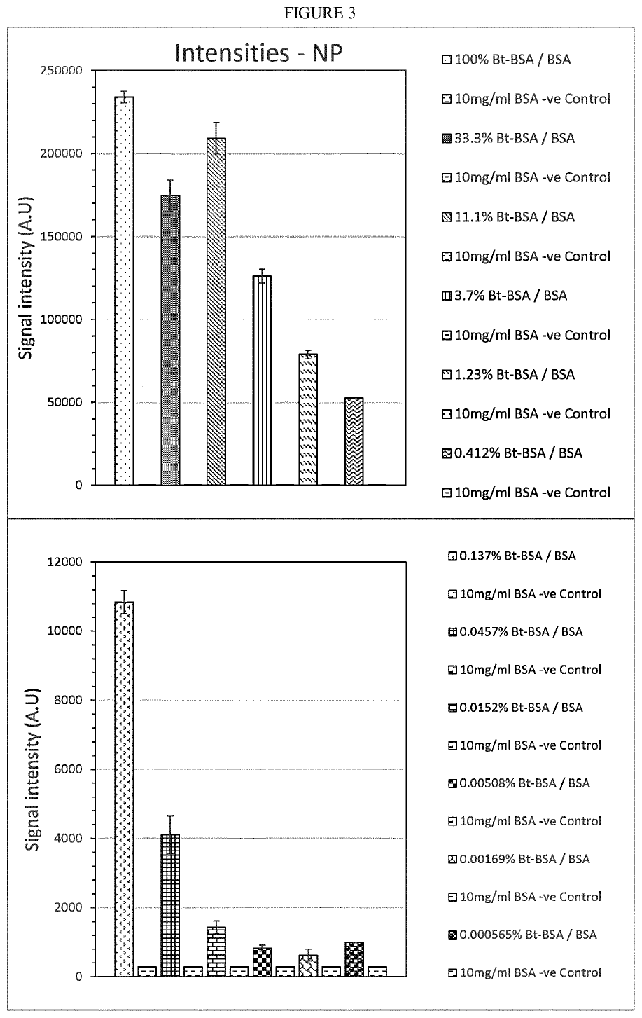

Results illustrated in FIG. 3 show the blank corrected positive intensities for the NP-containing wells collected using a 570-20 nm bandpass filter.

With the exception of the outlier data point of the 33% biotin-BSA, there is a trend of reducing the positive intensity with reduction of analyte on the surface.

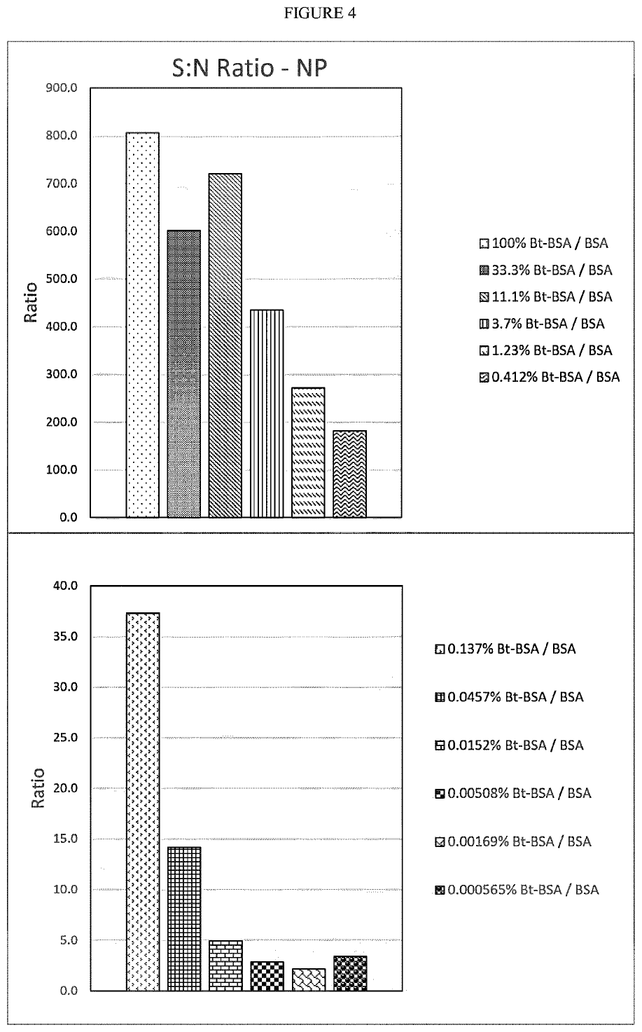

The negative controls were calculated as the blank-corrected average of cells G1-G4 for nanoparticle data. The negative controls are low, indicating little non-specific binding is occurring. As the signal to noise (S:N) ratio in this experiment, therefore, is governed almost exclusively by the impact on positive intensity, the S:N ratio drops proportionally to the reduction in positive intensity. This is clear from FIG. 4, which shows the S:N ratio at different analyte levels.

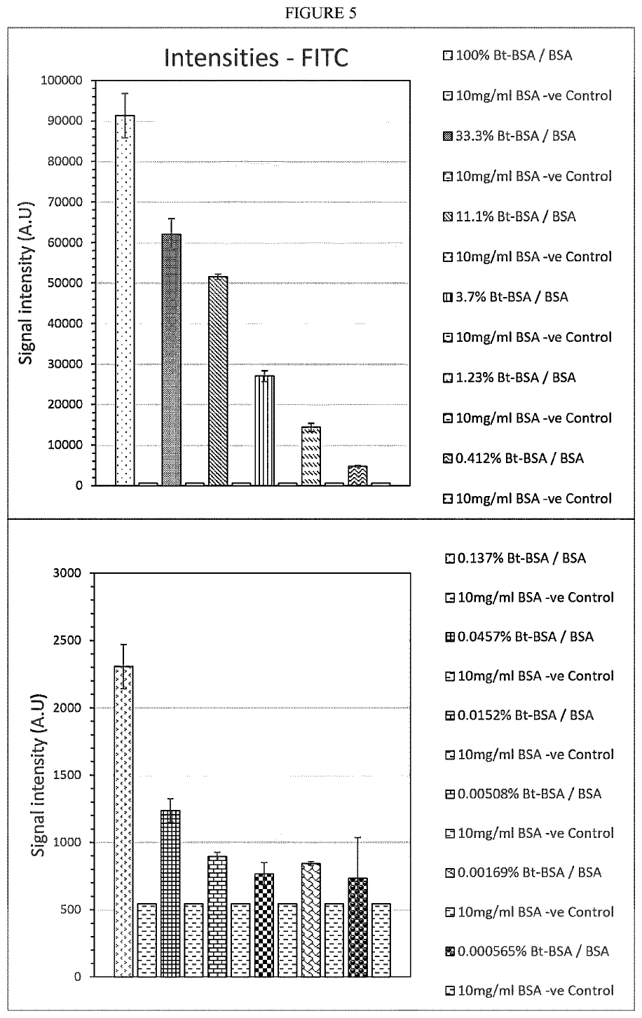

FIG. 5 is a graph showing the blank corrected positive intensities for FITC-containing wells collected using the 530-20 nm bandpass filter. Similarly to the nanoparticle data, the negative controls are the blank-corrected average of cells G6-G9.

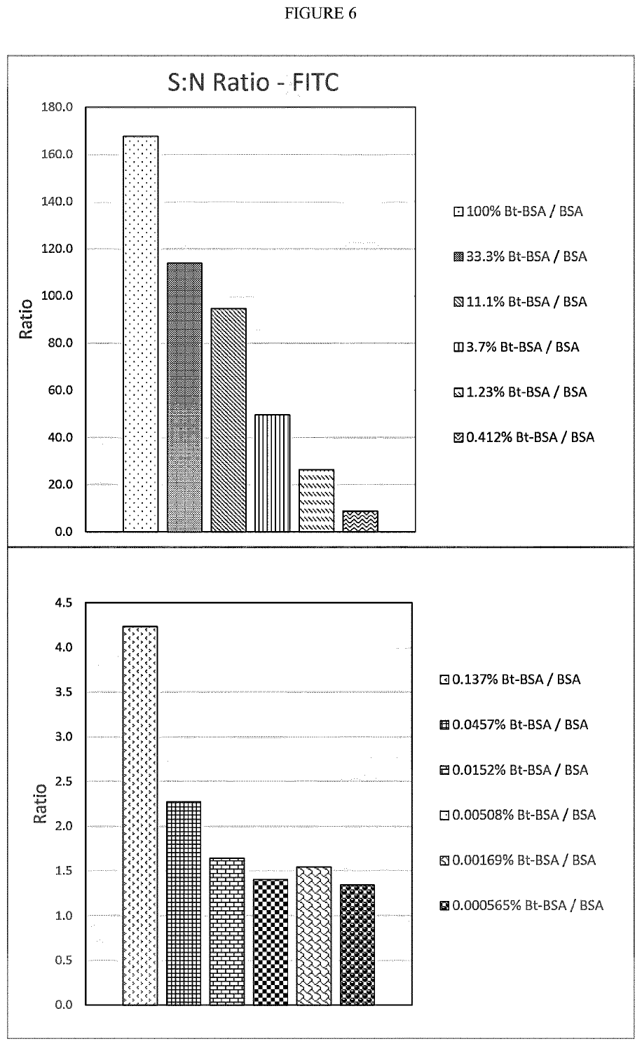

As with the NPs there is a clear trend of reducing the positive intensity with the reduction in analyte density. Similarly, the S:N ratio is reduced proportionally to the reduction in positive intensity (FIG. 6).

Due to the improved relative brightness of the NPs, the signal to noise ratio is significantly higher for the NPs vs FITC.

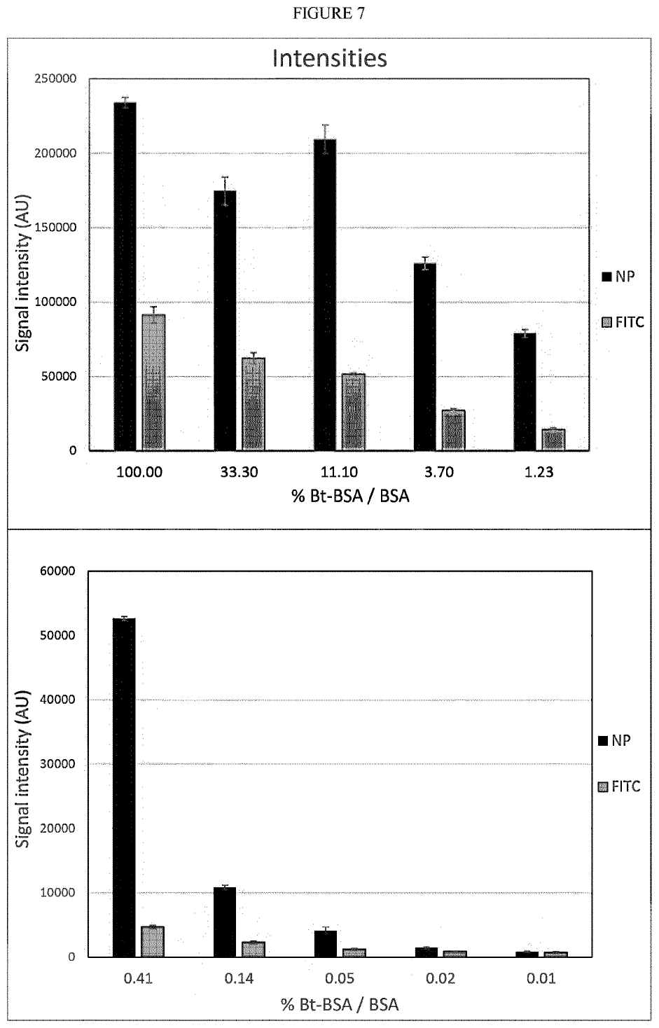

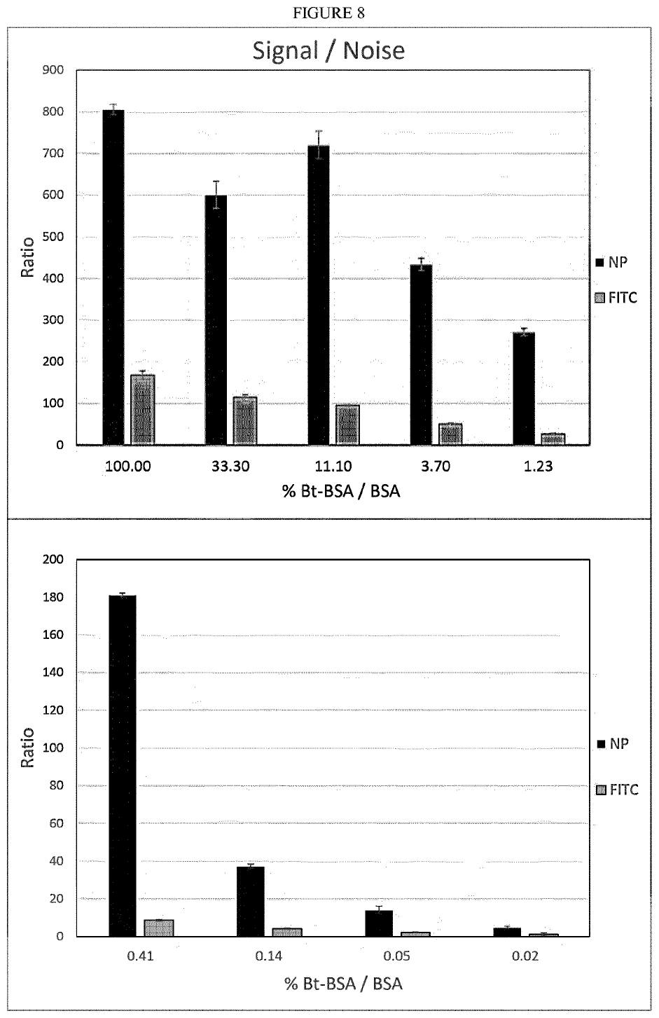

Graphs showing a comparison of the relative intensities (FIG. 7) and relative S:N ratio (FIG. 8) of the NPs and FITC on their respective filter sets shows that because the NPs are significantly brighter the drop in intensity, and hence S:N ratio, is less for the NPs than for FITC as the amount of analyte on the surface reduces (i.e. as the proportion of biotinylated surface is reduced).

Indeed, as the amount of analyte reduces, the FITC intensity becomes barely higher than the negative controls. Hence, the signal to noise ratio drops to approximately 1.5 by 0.015% biotinylated surface (i.e. 0.008 μg/ml biotin-BSA), as the limit of detection is approached.

In contrast, the NPs maintain a signal to noise ratio of greater than 2 even when the analyte surface density is 5.6×10-4% and, indeed, a measurable signal is still detectable at this analyte density.

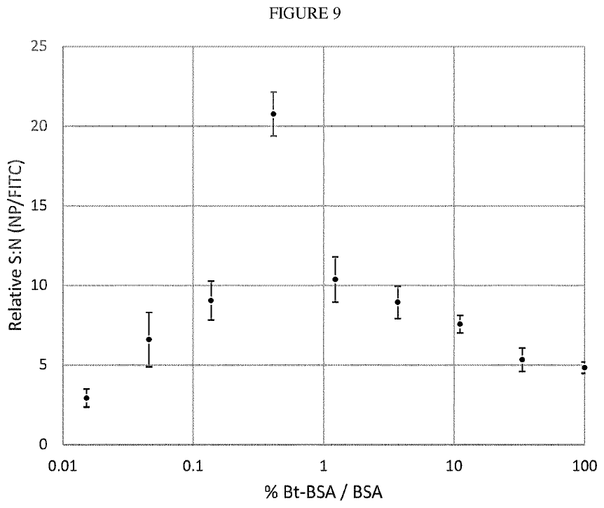

FIG. 9 is a graph showing the relative signal to noise for NPs vs FITC at different analyte density. This graph clearly shows that the relative performance of NPs vs FITC increases as the analyte density approaches 1%.

### Example 2

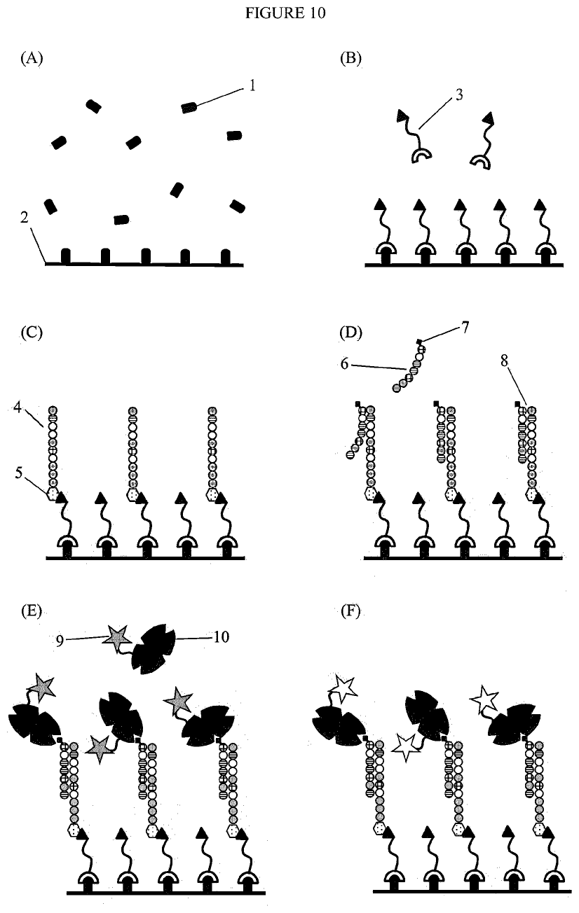

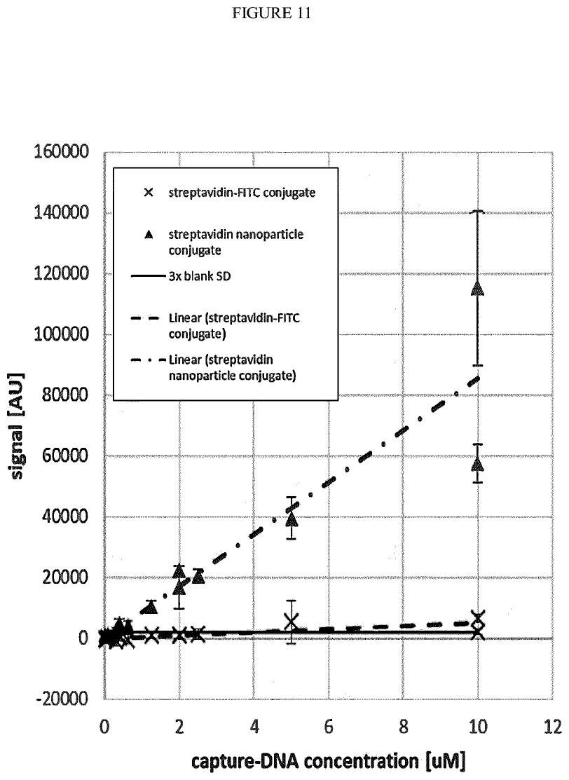

The experimental set up used in Example 2 is shown in FIG. 10.

FIG. 10A shows the application of a surface attachment polymer 1 to a glass surface 2.

In FIG. 10B, the surface is incubated with surface spacer group 3 which binds to the surface attachment polymer 1.

In FIG. 10C, the surface is incubated with single stranded ‘capture’ DNA 4, comprising a conjugated attachment group 5 which binds to the surface spacer group 3.

In FIG. 10D, the surface is incubated with a single stranded oligonucleotide 6 comprising a nucleotide to which a ligand 7 is conjugated. In the example detailed below, the ligand is biotin. The single stranded oligonucleotide 6 has a complementary sequence to the capture DNA strand 4, and thus the capture DNA strand 4 and complement strand 6 hybridise to form double stranded DNA 8 comprising a ligand-conjugated nucleotide.

In FIG. 10E, the surface is incubated with a marker comprising a fluorescent label 9 conjugated to a biomolecule 10 that has a high affinity for the ligand 7. In the example detailed below, the biomolecule is streptavidin 10, and the fluorescent label 9 comprises either a nanoparticle (NP), or FITC. As a result of a high affinity interaction between the ligand and biomolecule, the marker binds to the double stranded DNA 8. Thus, a marked nucleotide is formed, comprising a nucleotide conjugated to a light emitting marker by a linker composed of a ligand-biomolecule (in this example, biotin-streptavidin) coupling.

In FIG. 10F, any unbound marker is removed from the surface, and the surface is imaged to detect fluorescence from the fluorescent label 9.

Sample Preparation

Test samples comprising a fluorescent marker conjugated to streptavidin were prepared.

The fluorescent marker was either a nanoparticle (NP), or FITC.

The NPs have a significantly higher brightness than FITC. The markers were used in the amounts that had been previously determined to provide the best signal to noise ratio, which was 0.125 mg/ml NPs and 2.5 μg/ml of streptavidin-FITC.

Sample 1—Streptavidin-Conjugated Nanoparticles

1. 1 mg biotinylated NP (which was Particle 1 as used in Example 1) was resuspended in 1 ml borate buffer, as described in Example 1, containing 10 mg/ml BSA.
2. 50 μL of 7 mg/ml streptavidin was added to the resuspended NP solution, mixed, and incubated for 60 mins at room temp.
3. A streptavidin-spiked BSA-Borate buffer solution was prepared containing 0.03 mg/ml streptavidin in borate buffer containing 10 mg/ml BSA.
4. The streptavidin-NPs were resuspended in the streptavidin-spiked BSA-Borate buffer and sonicated.
5. The NPs were washed and resuspended in 97 μL of the streptavidin-spiked 10 mg/ml BSA in Borate buffer solution and 3 μL NaN3 (0.1 M).
6. The solid content of the NP preparation was calculated using a standard method.
7. Based on the determined solid content of the preparation, an NP solution was prepared containing 0.125 mg/ml NP in borate buffer containing 10 mg/ml BSA.

Sample 2—Streptavidin-FITC

A solution was prepared containing 2.5 μg/ml of commercially obtained Streptavidin-FITC in 3 ml borate buffer containing 10 mg/ml BSA.

Preparation of DNA Attachment Solution Serial Dilution

1a. In some experiments, 40ul of DBCO-TEG-10T-p5 solution (having a sequence: 5′ tri-ethylene glycol-DBCO-TTTTTTTTTTGAATGATACGGCGACCACCGA) was added to 1960 ul of DNA immobilisation buffer (140 nm sodium phosphate buffer pH 8.5 with 300 mM NaCl) to produce a 10 uM DNA solution. 300ul of the 10 uM DNA solution was then added to 1200ul of DNA immobilisation buffer to produce a 2 uM DNA solution. A serial dilution was then performed to produce 0.4, 0.08, and 0.016 uM DNA solutions.

1b. In other experiments, 20ul of DBCO-TEG-10T-p5 solution (having the same sequence as above) was added to 1980 ul of DNA immobilisation buffer to produce a 5 uM DNA solution. 1000ul of the 5 uM DNA solution was then added to 1000ul of DNA immobilisation buffer to produce a 2.5 uM DNA solution. A serial dilution was then performed to produce 1.25, 0.625, and 0.325 uM DNA solutions.

2. 5 uM DBCO-PEG-biotin solution (comprising commercially obtained Biotin-dPEG12-DBCO) was also produced.

Assay Plate Preparation

1. 90ul of 20 mM Amine-PEG4-Azide (14-Azido-3,6,9,12-tetraoxatetradecan-1-amine) in Sodium phosphate buffer was added to each well of a 96 well plate and plate was incubated for 30 minutes at room temp.

2. The Amine-PEG4-Azide was removed and the wells washed with sodium phosphate buffer.

3. The plate was prepared as follows (“buffer” refers to DNA immobilisation buffer) and incubated at 50C for 60 mins (example shown is for the serial dilution based on 10 uM DNA solution).

| | 1 | 2 | 3 | 4 | 5 | 6 | 7 | 8 | 9 | 10 | 11 | 12 |
|---|---|---|---|---|---|---|---|---|---|---|---|---|
| | DBCO-PEG-biotin | | 10 uM DNA | | 2 uM DNA | | 0.4 uM DNA | | 0.08 uM DNA | | 0.016 uM DNA | |
| A | DBCO- | DBCO- | 10 uM | 10 uM | 2 uM | 2 uM | 0.4 uM | 0.4 uM | 0.08 uM | 0.08 uM | 0.016 uM | 0.016 uM |
| B | PEG | PEG | DNA | DNA | DNA | DNA | DNA | DNA | DNA | DNA | DNA | DNA |
| C | biotin | biotin | | | | | | | | | | |
| D | | | | | | | | | | | | |
| E | buffer | buffer | | | | | | | | | | |
| F | buffer | buffer | | | | | | | | | | |
| G | buffer | buffer | | | | | | | | | | |
| H | buffer | buffer | buffer | buffer | buffer | buffer | buffer | buffer | buffer | buffer | buffer | buffer |

6. All wells were washed with PBS buffer.

7. A 10 uM solution of Primer 1.5B (5′ Biotin—p5 sequence), having a sequence complementary to DBCO-TEG-10T-p5, in hybridisation buffer, was prepared. 60ul of this solution was added to each of the wells labelled “primer” in the diagram below. Hybridisation buffer was added to the wells labelled “buffer”:

| | 1 | 2 | 3 | 4 | 5 | 6 | 7 | 8 | 9 | 10 | 11 | 12 |
|---|---|---|---|---|---|---|---|---|---|---|---|---|
| A | buffer | buffer | Primer | Primer | Primer | Primer | Primer | Primer | Primer | Primer | Primer | Primer |
| B | buffer | buffer | Primer | Primer | Primer | Primer | Primer | Primer | Primer | Primer | Primer | Primer |
| C | buffer | buffer | Primer | Primer | Primer | Primer | Primer | Primer | Primer | Primer | Primer | Primer |
| D | buffer | buffer | Primer | Primer | Primer | Primer | Primer | Primer | Primer | Primer | Primer | Primer |
| E | buffer | buffer | buffer | buffer | buffer | buffer | buffer | buffer | buffer | buffer | buffer | buffer |
| F | buffer | buffer | buffer | buffer | buffer | buffer | buffer | buffer | buffer | buffer | buffer | buffer |
| G | buffer | buffer | buffer | buffer | buffer | buffer | buffer | buffer | buffer | buffer | buffer | buffer |
| H | buffer | buffer | buffer | buffer | buffer | buffer | buffer | buffer | buffer | buffer | buffer | buffer |

8. All wells were washed with PBS buffer.

9. 60 ul of the samples to be tested was added to each well as indicated in the table below. To the wells labelled “blank” was added 60 ul of 10 mg/ml BSA in borate buffer. The plate was then incubated for 30 minutes.

| | 1 | 2 | 3 | 4 | 5 | 6 | 7 | 8 | 9 | 10 | 11 | 12 |
|---|---|---|---|---|---|---|---|---|---|---|---|---|
| A | Sample 1 | Sample 2 | Sample 1 | Sample 2 | Sample 1 | Sample 2 | Sample 1 | Sample 2 | Sample 1 | Sample 2 | Sample 1 | Sample 2 |
| B | | | | | | | | | | | | |
| C | | | | | | | | | | | | |
| D | | | | | | | | | | | | |
| E | | | | | | | | | | | | |
| F | | | | | | | | | | | | |
| G | | | | | | | | | | | | |
| H | Blank | Blank | Blank | Blank | Blank | Blank | Blank | Blank | Blank | Blank | Blank | Blank |

10. All wells were washed with borate buffer and then dried.

11. The plate was then imaged and fluorescence detected using an excitation wavelength of 475-30 nm (i.e. 460-490 nm) and an emission wavelength of 575-100 nm (i.e. 525-625 nm), using standard methods.

### Experimental Overview

| | 1 | 2 | 3 | 4 | 5 | 6 | 7 | 8 | 9 | 10 | 11 | 12 |
|---|---|---|---|---|---|---|---|---|---|---|---|---|
| | DBCO-PEG-biotin | | 10 uM DNA | | 2 uM DNA | | 0.4 uM DNA | | 0.08 uM DNA | | 0.016 uM DNA | |
| □ | 1 | 2 | 1 | 2 | 1 | 2 | 1 | 2 | 1 | 2 | 1 | 2 |
| A | P | P | P | P | P | P | P | P | P | P | P | P |
| B | P | P | P | P | P | P | P | P | P | P | P | P |
| C | P | P | P | P | P | P | P | P | P | P | P | P |
| D | P | P | P | P | P | P | P | P | P | P | P | P |
| E | N | N | N | N | N | N | N | N | N | N | N | N |
| F | N | N | N | N | N | N | N | N | N | N | N | N |
| G | N | N | N | N | N | N | N | N | N | N | N | N |
| H | Blank | Blank | Blank | Blank | Blank | Blank | Blank | Blank | Blank | Blank | Blank | Blank |

“P” represents positive sample
“N” represents negative control, and
“1” and “2” represent Sample 1 and Sample 2 respectively.

Results

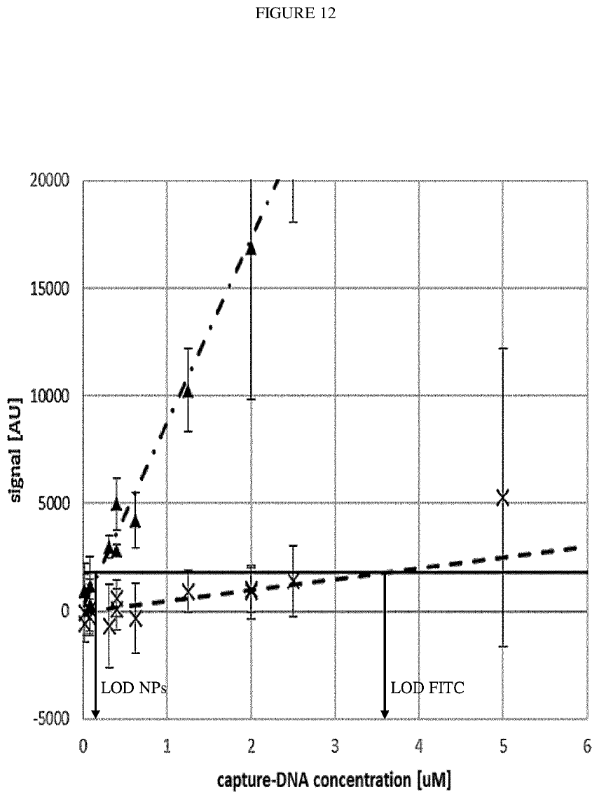

The results are shown in FIGS. 11 and 12. The data shown was obtained from three independent experiments.

FIG. 11 is a graph showing fluorescent signal intensity [AU] of the indicated markers at different concentrations of capture DNA strands. FIG. 12 shows an excerpt from FIG. 11.

In both FIGS. 11 and 12, the streptavidin conjugated nanoparticle markers are shown using triangles, and streptavidin-FITC markers are shown using crosses.

The fluorescent signal intensity (binding signal) was derived as follows:

Binding signal=blank corrected positive well signal—blank corrected negative well signal

The minimum detectable binding signal was defined as 3× the standard deviation of the blanks. The limit of detection (LOD) is the capture-DNA concentration corresponding to the minimum detectable binding signal. This value is obtained from the calibration curve (for calibration curve y=mx+q, LOD=(3*SD of the blank−q)/m).

The linear fit of signal intensity for conjugated nanoparticles (dashed line) was y=8531×+276, R²=0.87, and the linear fit for conjugated FITC-streptavidin (dash dotted line) was y=507×−39, R²=0.6686. The signal intensity threshold (continuous line) for the limit of detection (LOD) was calculated as 3 times the standard deviation of the blank value. Error bars represent the standard deviation of repeated wells on a single plate.

Arrows represent the visual representation of the LOD calculation as the concentration at which the signal intensity reaches the LOD signal intensity threshold for the conjugated nanoparticles (LOD NPs) and the FITC-streptavidin conjugate (LOD FITC).

Table 2 shows the LOD for the different markers and the LOD ratio.

| | LOD [uM] |
|---|---|
| Streptavidin-FITC | 3.6 |
| Streptavidin-nanoparticles | 0.18 |
| Ratio:FITC/NPs | 20 |

Using a brighter light emitting marker, in this case NPs, provides a much lower limit of detection than conventional fluorescent labels.

As a consequence, in the context of DNA sequencing, for a specific lower limit of signal detection, fewer copies (replicates) of the cloned DNA template fragments are required in order to provide a detectable signal. As a result, the use of a brighter dye allows the sequencing of longer DNA strands than is possible when conventional fluorescent labels are used. This is because the use of fewer cloned DNA template fragments in a cluster provides improved error detection and error elimination.

The use of a small number of cloned DNA template fragments in a cluster allows the improved detection of pre- and post-phasing errors arising as a result of the loss of uniformity in the location of the test nucleotide in the strands of a cluster. The fewer the number of cloned DNA template fragments, the larger the difference in signal arising as a result of the loss of uniformity. This increased difference in signal intensity can be detected and the necessary compensation and/or correction can then be applied.

In addition, the use of a brighter light emitting marker provides improved signal to noise ratio. Signal to noise ratio would be expected to be independent of brightness because specific and non-specific binding (i.e. positive and negative signals) would be expected to increase proportionally with increased brightness. The level of non-specific binding provided by high brightness light emitting markers, such as NPs, is significantly lower than expected, and as a proportion, is much lower than the level of non-specific binding seen when conventional fluorescent labels are used. The resulting improved signal to noise ratio provides further advantages such as improved limit of detection and signal correction.

1. A method of determining whether a test nucleotide comprises a base complementary to the next base of a template strand immediately downstream of a primer in a primed template nucleic acid molecule, the method comprising the steps of:
(a) providing a primed template nucleic acid molecule;
(b) providing a marked nucleotide comprising the test nucleotide conjugated to a light emitting marker by a linker, wherein the light emitting marker comprises a light-emitting particle;
(c) contacting the primed template nucleic acid molecule with a reaction mixture that comprises a polymerase and the test nucleotide, to thereby incorporate the test nucleotide into the primed strand of the primed template only if the test nucleotide comprises a base complementary to the next base of the template strand; and
(d) detecting light emitted by the light emitting marker, wherein the detection of light identifies the incorporation of the test nucleotide into the primed strand, and thereby indicates that the test nucleotide comprises a base complementary to the next base of the template strand;
wherein step (b) occurs before step (c), such that the test nucleotide is present in the reaction mixture in the form of a marked nucleotide, or
wherein step (c) occurs before step (b), such that the marked nucleotide comprising the test nucleotide is formed after incorporation of the test nucleotide into the primed strand.

2. The method of claim 1, wherein step (c) occurs before step (b), and the method comprises:
(a) providing a primed template nucleic acid molecule;
(b) contacting the primed template nucleic acid molecule with a reaction mixture that comprises a polymerase and a test nucleotide, to thereby incorporate the test nucleotide into the primed strand of the primed template nucleic acid molecule only if the test nucleotide comprises a base complementary to the next base of the template strand;
(c) conjugating the test nucleotide to a light emitting marker using a linker to form a marked nucleotide, wherein the light emitting marker comprises a light-emitting particle; and
(d) detecting light emitted by the light emitting marker, wherein the detection of light identifies the incorporation of the test nucleotide into the primed strand, and thereby indicates that the test nucleotide comprises a base complementary to the next base of the template strand.

3. The method of claim 1, wherein the method is performed using an array comprising clusters of cloned template fragments, each cloned template fragment representing a primed template nucleic acid molecule.

4. The method of claim 3, wherein each cluster comprises:
(a) less than 1000 primed template nucleic acid molecules;
(b) less than 100 primed template nucleic acid molecules; and/or
(c) less than 10 primed template nucleic acid molecules.

5. The method of claim 1, wherein the method does not comprise the use of clusters of cloned template fragments, and the method comprises the use of a single copy of each primed template nucleic acid molecule.

6. The method of claim 1, wherein the linker is a cleavable linker and the method further comprises step (e) cleaving the linker to dissociate the light emitting marker from the test nucleotide.

7. The method of claim 1, wherein the linker comprises a flexible spacer.

8. The method of claim 1, wherein the linker comprises a stable complex between a ligand and a biomolecule.

9. The method of claim 8, wherein:
(a) the ligand is an antigen and the biomolecule comprises an antigen-binding fragment or an antibody; or
(b) the ligand is biotin and the biomolecule is selected from avidin, streptavidin, neutravidin and recombinant variants thereof.

10. The method of claim 1, wherein the light-emitting marker is a light-emitting particle having a light-emitting core containing or consisting of a light-emitting polymer.

11. (canceled)

12. The method of claim 1, wherein the nucleotide further comprises a terminator or reversible terminator moiety.

13. The method of claim 1, wherein the light emitting marker has a brightness of at least 3×10⁶ cm⁻¹M⁻¹.

14. The method of claim 1, wherein the test nucleotide is attached to a ligand by a cleavable linker, and the light emitting marker comprises a biomolecule that is capable of forming a stable complex with the ligand, wherein the marked nucleotide is formed by contacting the ligand with the biomolecule thereby forming a stable complex between the ligand and biomolecule.

15. The method of claim 14, wherein contacting the ligand with the biomolecule to form a stable complex occurs after incorporation of the test nucleotide into the primed strand.

16. The method of claim 14, wherein the test nucleotide is present in the reaction mixture in the form of a marked nucleotide comprising the test nucleotide attached to the light emitting marker by a cleavable linker.

17. The method of claim 1, wherein the reaction mixture comprises a plurality of different species of test nucleotides, such as two, three, or four different species of test nucleotides, each comprising a different light emitting marker arranged to emit light at a different wavelength, wherein detecting light emitted by the light emitting marker of one of the plurality of different species of test nucleotides identifies the incorporation of that particular test nucleotide into the primed strand, and thereby indicates that the particular test nucleotide comprises a base complementary to the next base of the template strand.

18. A marked nucleotide comprising a nucleotide attached to a light emitting marker by a cleavable linker, wherein the light emitting marker is a light-emitting particle having a light-emitting core containing or consisting of a light-emitting polymer.

19. The marked nucleotide of claim 18, wherein the light emitting marker has a brightness of at least 3×10⁶ cm⁻¹M⁻¹.

20. (canceled)

21. The marked nucleotide of claim 18, wherein the light-emitting core contains the light-emitting polymer and a matrix material.

22. (canceled)

23. The marked nucleotide of claim 18, wherein the nucleotide further comprises a terminator or reversible terminator moiety.
